(19) United States
(12) Patent Application Publication
(10) Pub. No.: US 2023/0337668 A1
(43) Pub. Date: Oct. 26, 2023

(54) COMPOSITION

(71) Applicant: GIVAUDAN SA, VENIER (CH)

(72) Inventors: Simona BIRTIC, Cavaillon (FR); Ana Paula DO ESPIRITO SANTO, Avignon (FR); Michael LAGUERRE, Le Pontet (FR); François-Xavier Henri PIERRE, Saint Saturnin les Avignon (FR)

(21) Appl. No.: 18/023,944

(22) PCT Filed: Sep. 15, 2021
(86) PCT No.: PCT/EP2021/075315

§ 371 Date: Feb. 28, 2023

(30) Foreign Application Priority Data
Sep. 17, 2020 (GB) 2014665.0
Sep. 17, 2020 (GB) 2014669.2

(51) Int. Cl. A01P 3/00; A23L 3/3544; A23B 4/20; A01P 1/00; A23L 3/3508; A01N 37/40; A01N 65/22; A01N 43/90; A01N 65/08; A01N 65/40; A23L 3/3472; A01N 43/16; A01N 65/36

(52) U.S. Cl. CPC A01N 37/40; A01N 43/16; A01N 43/90; A01N 65/08; A01N 65/22; A01N 65/36; A01N 65/40; A01P 1/00; A01P 3/00; A23B 4/20; A23L 3/3472; A23L 3/3508; A23L 3/3544

(57) ABSTRACT

The present invention relates to preservative and/or antimicrobial compositions comprising at least one saponin and at least one phenolic diterpene.

### FIELD OF THE INVENTION

The present invention relates to preservative and/or antimicrobial compositions.

### BACKGROUND OF THE INVENTION

Microbial spoilage of food and beverages is a concern in the food industry today. Food and beverage matrices have varying degrees of sensitivity to microbiological spoilage depending on intrinsic factors of the matrix, such as pH, nutrient content (e.g., juice, vitamin, or micronutrient content; e.g. sauces fats, protein content, sugar level), carbonation level, Brix, dry matter level, water quality (e.g., alkalinity and/or hardness), and preservatives. Spoilage events occur when microorganisms are able to overcome the foods beverage’s intrinsic factors and grow. The microorganisms’ ability to-overcome these hurdles can be influenced by, among other things, initial contamination level, temperature and package integrity of the beverage against carbonation loss, i.e., in the case of carbonated soft drinks.

Microbiological spoilage can result from one or more yeasts, bacteria, and/or mold microorganisms. For example, yeasts and bacteria are capable of spoiling emulsified and non emulsified sauces such as mayonnaise, ketchup, vinaigrette, range, carbonated and non-carbonated beverages such as fruit drinks, teas, coffees, enhanced waters, etc. The ability of yeasts and certain bacteria to grow anaerobically enables their growth in carbonated beverages, while molds are restricted to aerobic metabolism, and therefore do not grow. See Stratford, M. et al., Fruit Juices, Fruit Drinks, and Soft Drinks, In The Microbiological Safety & Quality of Food (eds. B. M. Lund, T. C. Baird-Parker, and G. W. Gould, Aspen Publishers 2000). Typically, spoilage by yeasts manifests itself as fermentation with gas and ethanol production, as well as sedimentation, off-flavors and odors, and loss of cloud or emulsion stability. Bacteria tend to produce off-flavors and odors with associated sedimentation. On the other hand, molds may survive but generally are not capable of growth in low oxygen environments and thus, do not spoil carbonated soft drinks except when carbonation is diminished. Mold spoilage of non-carbonated beverages, however, can occur and may be evident after mold mycelial growth, by floating globules, clumps or surface pellicles.

Although yeasts such as Saccharomyces, Zygosaccharomyces, Candida, and Dekkera spp. are often responsible for spoilage incidents in common beverages, acidophilic bacteria such as Lactobacillus, Leuconostoc, Gluconobacter, and Zymomonas spp. and molds like Penicillium and Aspergillus spp. can also spoil cold-filled beverages. Spores of acidophilic, thermophilic bacteria such as Alicyclobacillus spp. and heat resistant mold spores of Byssochlamys and Neosartorya spp. can survive pasteurization and may spoil non-carbonated hot-filled products such as sport drinks and teas. Packaged waters are susceptible to growth by molds as well.

Protection against microbiological spoilage of beverages can be achieved using chemical preservatives and/or processing techniques such as hot filling, tunnel pasteurization, ultra-high temperature treatment (UHT) or pasteurization followed by aseptic packaging, and/or pasteurization followed by chilling the beverage. Generally, beverages with a pH<4.6 can be chemically preserved, heat processed, and filled into packages such that the product is not re-contaminated. For example, process techniques such as cold filling with chemical preservatives or pasteurization followed by cold-filling may be used to preserve this type of beverage. In a similar manner, this same beverage may be processed using non-preserved techniques such as hot filling, tunnel pasteurization, pasteurization followed by aseptic filling or even requiring the beverage to be chilled, i.e., under refrigeration following the pasteurization step. Beverages having a pH≧4.6 must be processed such that spores are destroyed using ultra-high temperatures followed by aseptic filling into packages or retorting sealed packages of product.

However, use of such methods impact the texture, colour and taste of food or beverages. The need is to avoid harsh treatments and keep food or beverage preserved from microbial spoilage. This may be achieved by using preservation ingredients.

Current preservation systems for acidic, shelf-stable carbonated and non-carbonated soft drinks or emulsified and non-emulsified sauces rely on chemically synthesised preservatives (e.g., benzoic and/or sorbic acid). Benzoic and sorbic acids (and salts thereof) effectively inhibit yeast, bacteria, and molds with some exceptions. Benzoic and sorbic acids in food and beverages exist in equilibrium between their dissociated and undissociated forms which is dependent upon the dissociation constant of the acid (pKa) and the beverage pH. The pKa for benzoic acid is 4.19 and the pKa of sorbic acid is 4.76. A food or beverage pH below the pKa of the particular acid pushes the equilibrium towards the undissociated form. The undissociated form is more efficacious against microorganisms, and therefore, weak acid preservatives may be most effective in the low pH range.

However, currently the industry wishes to move away from the use of chemically produced antimicrobials, such as benzoic or sorbic acids.

Moreover, weak acid preservation systems have limitations. Genetic adaptation and subsequent resistance by microorganisms may be one of the biggest concerns. (Piper, P. et al., Weak Acid Adaptation: The Stress Response that Confers Yeasts with Resistance to Organic Acid Food Preservatives, 147 Microbiol. 2635-2642 (2001)). Certain yeasts, such as Z. bailii, Z. bisporus, C. krusei, and S. cerevisiae, have specific genes that enable them to resist the weak acid preservatives and grow, despite their presence and regardless of the co-presence of EDTA or SHMP. Some bacteria, such as Gluconobacter spp., are also thought to be preservative resistant. The levels of weak acids necessary to overcome this resistance have been shown to be far beyond regulatory limits on use levels. Spoilage of preserved sauces, teas, juice-containing beverages, and carbonated beverages is commonly due to preservative-resistant yeasts.

Weak acids are also known to impart a throat or mouth burn when used at high levels. Although there are certain shelf-stable foods and beverages where this may be acceptable, often this sensory perception is considered negative. In addition, non-government organizations and also some international government agencies have raised concerns regarding the use of weak acid preservatives in beverages and foods.

In addition, the other process techniques for low acid foods and beverages (i.e. with a pH≧4.6) have limitations. Such low acid food and beverages should be thermally treated to sufficiently destroy spores of Clostridium botulinum and Bacillus cereus. Examples of such processes include UHT and retort. Even after such processing, product should be handled in a way to prevent post-processing contamination. Research, however, suggests that there may still be various strains of spore forming microorganisms that can survive these different processing techniques. To that end, these processing techniques may not eliminate the potential for spoilage. Moreover, such techniques deplete the taste, the texture and the colour of food, disabling it from its initial freshness and appeal.

Natural preservatives having the capability of preserving food and beverages, or cosmetics may be desirable for consumers. Preservatives that could be labelled as natural could also eliminate hot-fill requirements for unpreserved shelf-stable preservative free food, beverages and cosmetics. Thus, it would be desirable to provide a natural preservative and/or a preservative system that inhibits growth of microorganisms to solve at least one of the above-mentioned limitations in the art.

Saponins are naturally occurring compounds and can be found in a variety of plants. For example, peanuts have from 1.3% to 1.6%, spinach root has about 4.7%, horse chestnut has about 3% to 6%, guar has about 10%, and asparagus has about 1.5% of saponins. ( Price et al., The Chemistry and Biological Significance of Saponins in Foods and Feeding Stuffs, 26 CRC Crit. Rev. Food Sci. Nutr. 27-135 (1987)). Despite those naturally occurring saponins in many plants used as human food, there are only two plant sources that are approved as food additives. The two are: Quillaja saponaria (triterpene-saponins) and Yucca schidigera (steroidal- saponins). These saponin-comprising extracts are currently regarded as generally recognized as safe (GRAS) products and are permitted to be used in food and beverages in the United Kingdom and United States and other regions. Moreover, Yucca extracts generally contain about 10% of dry weight saponins. (Oleszek, Wieslaw, et al., Steriodal Saponins of Yucca schidigera Roezel, 49 J. Agric. Food Chem. 4392 (2001))

Saponin-comprising extracts are known to have certain beneficial characteristics and uses such as foaming agents as used in U.S. Pat. No. 4,986,994, surfactants as used in U.S. Pat. Nos. 5,503,766 and 6,214,349, food flavorants as used in U.S. Pat. No. 5,804,239, agents in sanitary wipes as used in U.S. Pat. No. 6,734,157, and therapeutic agents as used in U.S. Publication No. 2004/0096527. In addition, Japanese Publication No. 2003009832 teaches a keeping improver particularly directed to inhibiting sprout growth of bacterial spores that contains an extract from saponin vegetation chosen from Sapindus mukurossi, a horse chestnut and asparagus as active agents. Studies contend that saponins are generally not absorbed in the digestive tract and thus, do not lead to serious toxicological problems, while oral toxicity of saponins has been estimated to be low. (Price et al., Chemistry and Biological Significance of Saponins in Foods and Feed-ingstuffs, 26 CRC CHt. Rev. Food Sci. Nutr. 27-135 (1987)).

Although saponin comprising extracts have been utilized in different capacities, the present inventors surprisingly discovered that saponin(s) in combination with Lamiaceae extracts extract can be used as a preservative in replacement of traditional preservative systems and/or can be used in conjunction with known preservatives to maintain microbial stability, microbial reduction, or enhanced stability or reduction or even to enhance microstability and may be used to reduce levels of traditional preservative systems.

### SUMMARY OF THE INVENTION

In a first aspect, the invention provides a composition (or first composition of the invention) comprising a) at least one Lamiaceae extract and/or b) at least one saponin.

In a second aspect, the invention provides a composition (or second composition of the invention) comprising a) at least one saponin and/or b) at least one phenolic diterpene.

In a third aspect, the invention provides a food or beverage product for humans or animals, a nutritional supplement, nutraceutical formulation, a dietary, a sports supplement, a fragrance or flavouring, a pharmaceutical or veterinary formulation, an oenological or cosmetic formulation comprising a first composition of the invention a second composition of the invention and/or a third composition of the invention.

In a further aspect, the invention provides the use of a first composition of the invention and/or a second composition of the invention as preservative and/or antimicrobial.

In a further aspect, the invention provides at least one saponin(s) for use as preservative and/or antimicrobial.

In a further aspect, the invention is related to a method for processing a food or beverage product for humans or animals, a nutritional supplement, a nutraceutical formulation, a fragrance or flavouring, a pharmaceutical or veterinary formulation, an oenological or cosmetic formulation comprising: applying to or incorporating into a food or beverage product for humans or animals, a nutritional supplement, a nutraceutical formulation, a fragrance or flavouring, a pharmaceutical or veterinary formulation, an oenological or cosmetic formulation, a first composition of the invention and/or a second composition of the invention.

In a further aspect, the invention relates to an antimicrobial composition comprising an effective amount of a phenolic diterpen (such as carnosic acid and/or carnosol) and/or at least one saponin (such as quillaja and/or yucca saponin(s)).

In a further aspect, the invention relates to an antibacterial, antifungal and/or antimold composition comprising an effective amount of a phenolic diterpen (such as carnosic acid and/or carnosol) and/or at least one saponin (such as quillaja and/or yucca saponin(s)).

The details, examples and preferences provided in relation to any one or more of the stated aspects of the present invention will be further described herein and apply equally to all aspects of the present invention. Any combination of the embodiments, examples and preferences described herein below in all possible variations thereof is encompassed by the present invention unless otherwise indicated herein, or otherwise clearly contradicted by context.

### DETAILED DESCRIPTION OF THE INVENTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

“Food”, “food product” and “foodstuff’ mean products that people or animals eat, such as, a solid, semi-solid or liquid (such as beverage) food stuff product known in the art.

As used herein, the term “beverage” or “beverage composition” refers to a liquid drink that is appropriate for human or animal consumption. Mention may be made, of beverages, but not limited to, for example, energy drinks, flavored water, fruit smoothies, sport drinks, fruit juices (e.g., juice drinks and full strength fruit juice), carbonated sodas/juices, shakes, protein drinks (e.g., dairy, soy, rice or other), meal replacements, drinkable dairy yogurts, drinkable soy yogurts, teas, coffees, cola drinks, fortified waters, low acid beverages as defined in 21 C.F.R §113, acidified beverages as defined in 21 C.F.R. §114, syrups, cordials, dilutables such as squashes, health drinks, functional beverages (e.g., nutraceuticals), nectars, tonics, horchata (i.e., vegetable and/or rice components made into a beverage), frozen carbonated beverages and frozen uncarbonated beverages.

“Fresh meat, fish, and poultry” means meat fish and poultry, entire carcasses, cut portions thereof, and ground portions thereof. Fresh meat, fish, and poultry includes both unprocessed meat, fish and poultry as well as meat, fish, and poultry that includes additives such as polyphosphates, salt, water, flavors, broths, added proteins, sugar, starches and the like which are incorporated into the meat, fish or poultry. It is important to distinguish fresh meat, fish or poultry which may contain these ingredients, from “processed” meat, fish and poultry which includes cured meat, fish and poultry, which may contain the same ingredients, but also contain one or more of the following: erythorbates, erythorbic acid, ascorbates, ascorbic acid, nitrites, nitrates or cultures. Fresh meat, fish and poultry are to be distinguished from, and as opposed to, and does not include cured meat, fish or poultry, known as processed meat, fish and poultry.

The section headings used herein are for organizational purposes only and are not to be construed as limiting the subject matter described. All documents, or portions of documents, cited in this application, including, but not limited to, patents, patent applications, articles, books, etc are hereby expressly incorporated by reference for the portions of the document discussed herein, as well as in their entirety.

### Compositions of the Invention

The inventors have surprisingly found that the combination of Lamiaceae extracts and at least one saponin (such as saponins from yucca and/or from quillaja saponin(s)) have an antimicrobial effect on different bacteria, yeast and molds. This can be observed on examples 1 to 6. The compositions of the invention have a synergistic antimicrobial effect even against very resistant species of food and beverage spoilage microorganisms such as Dekkera anomala as it is demonstrated on examples 5 and 6. Moreover the antimicrobial effect has been demonstrated at a broad range of concentration of the different components (such as 1%, 0,5%, 0,25%, 0,125%, 0.061% and 0.031% w/w).

According to the present invention, there is provided a composition, or first composition of the invention, comprising i) at least one Lamiaceae extract and/or ii) at least one saponin.

As used herein, the term “Lamiaceae extract” may refer to an extract from a plant of the Lamiaceae family (Lamiaceae material), such as rosemary, sage, oregano, thyme, mints, and the following genera: Salvia (such as Salvia Apiana and Salvia officinalis), Rosmarinus (such as Rosmarinus officinalis), Lepechinia, Oreganum, Thymus (such as Thymus vulgaris), Hyssopus and mixtures thereof.

The Lamiaceae material used for extracting the Lamiaceae extract can be any part of the plant such as leaves, roots, etc.

The Lamiaceae material may be processed before extraction, for example it can be washed, dried, milled or grounded, etc.

Particular solvents that may be used in the extraction process include water, alcohols (such as methanol, ethanol), acetone, ethyl acetate, hexane, dichloromethane, and any mixtures thereof, such as alcohol/water mixtures (such as mixtures of methanol and water). For example, the extraction solvents can be water, a water-alcohol mixture (from about 1% to about 99% alcohol in water. For example, from about 30% to about 75% alcohol in water, or from about 30% to about 50% alcohol in water, such as from about 35% or from about 40% alcohol in water), or alcohol. Particular alcohols that may be mentioned include ethanol (EtOH) and methanol (MeOH).

In particular embodiments, the extraction solvent may be a methanol-water mix, such as from about 30% to about 90% methanol in water, or from about 30% to about 50% methanol in water. For example, from about 50% or from about 80% ethanol in water. In a preferred embodiment the extraction solvent is ethanol-water mix with about 75% methanol and about 25% water.

The term “acetone extract” as used herein, refers to the extract obtained from any member of the Lamiaceae family (such as rosemary, salvia etc) when the extraction from the plant (particularly, leaves) has been performed using acetone as the only solvent.

The term “alcohol extract” as used herein, refers to the extract obtained from Lamiaceae when the extraction from the plant (particularly, leaves) has been performed using alcohol as the only solvent. For example, 100% methanol and/or 100% ethanol. The term “hydro-alcoholic extract’ as used herein, refers to the extract obtained from Lamiaceae (such as rosemary, salvia etc) when the extraction from the plant has been performed using a mixture of water and alcohol. For example, from about 1% to about 99% alcohol (e.g. ethanol, methanol) in water, such an extract would be termed a hydro-ethanolic extract.

A detailed procedure to prepare a Rosemary extract was described in the U.S. Pat. No. 5,859,293 (PCT W096/34534), which is incorporated herein by reference in its entirely.

For example, processes for extraction and isolation of extracts of the invention may comprise (or consist essentially/consist of) the following steps:
(i) extraction of Lamiaceae leaves (such as rosemary and /or salvia which may be ground) by a suitable solvent (such as acetone or ethanol);
(ii) evaporation of the solvent; and, if required
(iii) purification of the extract (e.g. by chromatography).

In certain embodiments, the temperature of extraction is in a range of from about 20° C. to about 100° C. In a particular embodiment, the temperature for extraction is in a range of from about 50° C. to about 70° C. Typically, the ratio of plant material to solvent mixture used in the extraction process varies from about 1 : 1 to about 1 :10 on a gram to milliliter basis, such as from about 1 :3 to about 1 :8. The incubation period (i.e. the period during which the plant material is in contact with the solvent) is typically from about 2 hours to about 24 hours.

Mechanical energy can be applied during the extraction process. Applying mechanical energy helps to homogenize the mixture, changes the physical structure of the starting biological material and increases the extraction yields of phenolic diterpens. The amount of mechanical energy applied in the method depends on at which step applied, the type of Lamiaceae material, the amount of the starting material used in the mixture, the pH of the mixture, and the temperature of the mixture. The amount of mechanical energy also can influence the amount of time needed to complete the extraction of the For example, the Lamiaceae material (such as rosemary and / or salvia) and the extraction solution (such as acetone or ethanol) may be mixed using techniques known in the art, for example using stirring, maceration, percolation or infusion, such as magnetic or mechanical stirring.

Stirring may be conducted at any suitable revolution per minute (rpm), for example, the stirring may be done from about 1 rpm or about 10 rpm or about 50 rpm to about 500 rpm. For mechanical stirring this may typically be done from about 1 rpm to 500 rpm, such as from about 10 rpm to about 200 rpm.

Devices for applying mechanical energy can be a pump, a refiner, a homogenizer, an extruder, a lobe pump, and/or a centrifugal pump. The mixture can be circulated in a closed-loop system that includes a pressure vessel (able to contain a heated solvent mixture), a reflux vessel, a heat exchanger, such as a shell and tube heat exchanger, and a pump for recirculating the heated mixture back to the vessel, allowing multiple passes through the pump in the system.

After the Lamiaceae material (such as rosemary and/or salvia leaves) and solvent have been incubated, the solvent is separated from residual Lamiaceae material by any suitable separation technique known in the art (like for example filtration).

Further filtration steps can be used. The solvent may be partially or totally removed by any method known in the art such as centrifugation, Rota vap, and any device allowing solvent evaporation or a liquid-liquid way of replacing the solvent.

Further filtration steps can be used. For example in a preferred embodiment, aqueous sodium carbonate (NaHCCh) may be added to dissolve carnosic acid and other organic acids, while base insoluble substances are precipitated out. The solution may be filtered to separate from solid, and the filtrate can be further concentrated under reduced pressure. In a further step, after finishing concentration is achieved, phosphoric acid (H3PO4) may added and the acid insoluble substances (including carnosic acid, carnosol, and carnosic derivatives) are precipitated from the concentrated solution. Additionally the result may be fileted and the solid precipitate may be subsequently separated from liquid and rinsed with water to remove impurities.

Sterilisation methods can be applied at any step of extraction.

In certain embodiments, the fist composition of the invention comprises (or consist essentially/consist of) a Salvia extract (such as Salvia Apiana and Salvia officinalis). In a preferred embodiment, the Salvia extract is enriched in phenolic diterpenes.

In certain embodiments, the fist composition of the invention comprises (or consist essentially/consist of) a Rosemary extract (such as Rosemary officinalis). In a preferred embodiment, the rosemary extract is enriched in phenolic diterpenes.

In certain embodiments, the Lamiaceae extract is enriched in phenolic diterpenes.

As used herein, the term “Phenolic diterpenes” may refer to carnosic acid, carnosol, methylcarnosate, and other phenolic diterpene derivatives (rosmanol, isorosmanol, 1 1, 12 -di-O- methylisorosmanol, 12-O-methylcarnosic acid, rosmanol-9-ethyl ether, circimaritin, Methylated monooxidized product of carnosic acid, genkwanin, epirosmanol, epiisorosmanol, carnosic acid derivative, epirosmanol ethyl ether, cryptotanshinone) and mixtures thereof.

For example, the composition of the invention comprises (or consist essentially/consist of) i) at least one Lamiaceae extract (such as rosemary and / or salvia extract) and ii) at least one saponin, wherein the Lamiaceae extract is enriched in phenolic diterpenes such as carnosic acid, carnosol, methylcarnosate, other phenolic diterpene derivatives such as rosmanol, isorosmanol, 11, 12 -di-O- methylisorosmanol, 12-O-methylcarnosic acid, rosmanol-9-ethyl ether, circimaritin, Methylated monooxidized product of carnosic acid, genkwanin, epirosmanol, epiisorosmanol, carnosic acid derivative, epirosmanol ethyl ether, cryptotanshinone and mixtures thereof.

In an embodiment, the first composition of the invention comprises at least one Lamiaceae extract (such as rosemary and/or salvia extract) comprising at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90%, at least about 95%, or at least about 99 wt % of one or more phenolic diterpenes such as the ones described before. In a preferred embodiment, the Lamiaceae extract(s) of the composition of the invention comprises carnosic acid and/or carnosol.

In a preferred embodiment, the first composition of the invention comprises at least one Lamiaceae extract (such as rosemary and/or salvia extract) comprising at least about 35% wt % of phenolic diterpenes (such as carnosic acid and/or carnosol).

In certain embodiments, the Lamiaceae extract (such as rosemary and/or salvia extract) may comprise (or consist essentially/consist of) the following phenolic diterpenes: carnosic acid and/or carnosol and the ratio between carnosic acid and carnosol is from 40:1 to 1:40, such as 30:1, 20:1, 10:1, 5:1 or 1:1.

In certain embodiments, the first composition may comprise (or consist essentially/consist of):
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w) of carnosic acid, such as from about 20% to about 80% w/w, preferably such as from about 30% to about 50 % w/w such as about 40% w/w; and/ or
b) from about 1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition(w/w), such as from about 1% to 20% w/w such as from about 2 % to about 10% w/w, preferably such as from about 3% to about 5% w/w, such as about 4% w/w.

For example, the first composition of the invention comprises (or consist essentially/consist of) i) at least one Lamiaceae extract (such as rosemary and / or salvia extract) and ii) at least one saponin saponin (such as quillaja and/or yucca saponin(s)). wherein the Lamiaceae extract(s) comprises (or consist essentially/consist of) carnosic acid and/or carnosol and the ratio between carnosic acid and carnosol is from 40:1 to 5:1, such as 30:1, 20:1, 10:1 or 5:1.

For example, the first composition of the invention comprises (or consists essentially/consist of):
i) at least one Lamiaceae extract (such as rosemary and / or salvia extract), and
ii) at least one saponin (such as quillaja and/or yucca saponin)
wherein the concentration of the following phenolic diterpenes in the final composition is:
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w) of carnosic acid, such as from about 20% to about 80% w/w, preferably such as from about 30% to about 50 % w/w such as about 40% w/w; and/ or
b) from about 1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition(w/w), such as from about 1% to 20% w/w such as from about 2 % to about 10% w/w, preferably such as from about 3 % to about 5% w/w, such as about 4% w/w.

In particular embodiments, the Lamiaceae extract (such as rosemary and/or salvia extract) may be:
substantially free of other plant material (e.g. free of plant cellulose);
substantially free of plant cells; and/or
substantially free of plant cellular matter,
substantially free of toxic components like pesticides, quintozene, aflatoxins, ochratoxin A, cadmium, arsenic, lead or mercury,
substantially free of solvents
substantially free of volatile oil components.

In a particular embodiment, the Lamiacea extract is an extract wherein a majority of the volatile oil components have been removed.

As used herein, the term “volatile oil components” may refer to compounds like essential oils such as: (-)-borneol, (-)-bornyl acetate, (-)-camphor, 1,8-Cineole (eucalyptol) and verbenone.

In certain embodiments the ration between the total % of phenolic diterpenes (such as carnosic acid and carnosol) / Total % of volatiles oil components (such as (-)-borneol, (-)-bornyl acetate, (-)-camphor, 1,8-Cineole (eucalyptol) and verbenone) is not less than 15.

As used herein, references to a material being “substantially free” of another material may refer to the material consisting of less than 1% by weight (e.g. less than 0.1%, such as less than 0.01% or less than 0.001%, by weight) of that other material-

Saponins are a group of naturally occurring glycosides, predominantly found in the plant kingdom. They comprise a non-carbohydrate aglycone coupled to sugar chain units. Saponins are divided in two groups: steroidal and triterpene saponins. Over 100 steroidal and an even higher number of triterpene saponins have been so far identified. (K. Hostettmann, & A. Marston, Saponins (Cam-bridge University Press 1995). The saponins of the present invention can be of natural origin or of synthetic origin. It may be one or more saponins from the same or different origin.

For example, the saponin(s) can be obtained or obtainable from plants such as soya, beans, peas, oat, Solanum and Allium species, tomato, asparagus, tea, peanut, spinach, sugar beet, yam, blackberry, liquorice root, primula root, senega root, Quillaja (such as Quillaja saponaria), Yucca (such as Yucca shidigera), and/or Gyposphila.

As used herein, the term “quillaja saponin(s)”, “yucca saponi(s), “oat saponin(s)” etc, means one or more saponins that can be obtained or obtainable from any of the members of the quillaja family, or the yucca family or any of the plants that contains saponins such as the ones described before. The quillaja saponin or the mixture of quillaja saponins (or the yucca saponin or the mixture of yucca saponins) can be of synthetic or natural origin.

As will be appreciated by the person skilled in the art, as used herein the term “obtainable from” means that the saponin(s) may be obtained from a plant or may be isolated from the plant, or may be obtained from an alternative source, for example by chemical synthesis or enzymatic production. Whereas the term “obtained” as used herein, means that the saponin(s) is directly derived from the plant. For example in certain embodiments, the saponin(s) can be a natural extract comprising saponin(s).

The at least one saponin, may be of natural or synthetic origin.

A “purified saponin(s)” means one or more saponins of natural or synthetic origin that have a concentration of at least about 80%, at least about 90%, at least about 95%, at least about 99%, at least about 99.9% of one or more saponins as described before (such as quillaja saponin(s) and /or yucca saponin(s)).

A “saponin(s) comprising extract” means any natural extract comprising at least one type of saponin as described before that may be derived from, e.g., but not limited to soya, beans, peas, oat, Solanum and Allium species, tomato, asparagus, tea, peanut, spinach, sugar beet, yam, blackberry, liquorice root, primula root, senega root, Quillaja (such as Quillaja sapona-ria), Yucca (such as Yucca shidigera), and/or Gyposphila. In certain embodiments the saponin is not a rosemary saponin.

According to the present invention, the at least one saponin may be derived from a single source or from multiple sources. According to the present invention, the at least one saponin comprising extract may be derived from a single source or from multiple sources.

Examples of yucca include, but are not limited to, Yucca aloifolia, Yucca angustissima, Yucca ar-kansana, Yucca baccata, Yucca baileyi, Yucca brevifolia, Yucca campestris, Yucca capensis, Yucca carnerosana, Yucca cernua, Yucca coahuilensis, Yucca constricta, Yucca decipiens, Yucca declinata, Yucca de-smetiana, Yucca data, Yucca endlichiana, Yucca faxoniana, Yucca filamentosa, Yucca filifera, Yucca flaccida, Yucca gigantean, Yucca glauca, Yucca gloriosa, Yucca grandiflora, Yucca harrimaniae, Yucca intermedia, Yucca jaliscensis, Yucca lacandonica, Yucca linearifolia, Yucca lu-minosa, Yucca madrensis, Yucca mixtecana, Yucca necopina, Yucca neomexicana, Yucca pallida, Yucca periculosa, Yucca potosina, Yucca queretaroensis, Yucca reverchonii, Yucca rostrata, Yucca rupicola, Yucca schidigera, Yucca schottii, Yucca sterilis, Yucca tenuistyla, Yucca thompsoniana, Yucca treculeana, Yucca utahensis, or Yucca valida. In certain preferred embodiments the yucca is Yucca schidigera.

The most abundant Y. schidigera stem/bark saponins are steroidal saponins. They differ in the structure of their aglycon, according to which, they are classified into spirostane- or furostane-type derivatives. Primary saponins are glycosides of three C-25 epimeric pairs of sapogenins: sar-sapogenin and smilagenin, markogenin and samogenin, gitogenin and neogotogenin. In both, spirostane and furostane derivatives, the C-3 carbohydrate chains are typically branched oligosaccharides with pentapyranosyl and/or hexopyranosyl units. As far as furostane bidesmosides are concerned, C-26 linked carbohydrate usually corresponds to a hexopyranose. It should be noted that derivatives of other sapogenins occur as minor compounds within Y. schidigera stem/bark. Classical methods presently used in measurement of steroidal saponins include spectrophotometric measurements, foam height measurements or gravimetric method. HPLC/ELSD technique is an accurate and reliable method that yields in results of appropriate repeatability and reproducibility.

Examples of quillaja include, but are not limited to, Quillaja brasiliensis, Quillaja lanceolata, Quillaja lancifolia, Quillaja molinae, Quillaja petiolaris, Quillaja poeppigii, Quillaja saponaria, Quillaja sellowiana, or Quillaja smegmadermos. In certain preferred embodiments the quillaja is Quillaja saponaria.

A person of ordinary skill in the art will appreciate that, as used herein, a plant name may refer to the plant as a whole, or to any part of the plant, such as the roots, stem or trunk, bark, leaves, flower, flower stems, or seeds or a combination thereof. These plant parts may be used fresh, or dried, and may be whole, pulverized, mashed, comminuted or ground up. Extracts from any part or parts of the plant are also contemplated.

Saponin containing extracts include juices, concentrate juices, dry juices and extracts obtained using solvents such as the ones described previously.

Saponins extracts can be obtained using similar extraction methods as described before for the Lamiaceae extracts. In a preferred embodiment the solvent is a methanol/water (for example 70:30 v/v) and the incubation time is of 24 hours at ambient temperature. Another extraction solvent can be only water. In a particular embodiment the quillaja containing material is incubated with water at a temperature from 50 to 100° C. (such as 50-60° C. or 100° C.). The extraction may be performed using a soxhlet apparatus or by maceration and filtration. The incubation time may be from some hours (like 10 hours) to 24 hours or more.

In certain embodiments, the Yucca (such as Y. schidigera) saponin comprising products or extracts are selected from a juice, a concentrated juice, a dry juice or a water extract.

Saponin comprising extracts may include other compounds that are not saponins such as naturally occurring glicocomponents, polyphenols, salts and sugars.

In certain embodiments, the purified saponin(s) or the “natural extract comprising saponin(s)” are from Yucca schidigera and/or Quillaja saponaria. Further, the at least one saponin or the saponin-comprising extract can be chosen from steroidal and triterpene saponins, and mixtures thereof. In a preferred embodiment, the at least one saponin (such as a purified saponin(s) or a saponin extract) is obtained or obtainable from Quillaja (such as Q. saponaria).

In another preferred embodiment, the at least one saponin (such as a purified saponin(s) or a saponin extract) is obtained or obtainable from Yucca (such as Yucca schidigera).

In another preferred embodiment, the at least one saponin (such as a purified saponin(s) or a saponin extract) is obtained or obtainable from Quillaja (such as Q. saponaria) and from Yucca (such as Yucca schidigera).

This application concerns Yucca or Quillaja genus extracts, juice or any other product comprising saponins, more precisely saponins corresponding to any Yucca or Quillaja saponin structure. Y. schidigera or Q. Saponaria extract or spray dryied extract in presence of inverted sugar or any other drying support that is well known in the art may be used in this application.

In certain embodiments of the first composition of the invention, the saponin(s) is a natural extract comprising saponin(s) (such as from Yucca schidigera and/or Quillaja saponaria) that comprises (or consist essentially/consist of) at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90%, at least about 95%, or at least about 99 w/w % of one or more saponin(s) as defined before (such as triterpenic and steroidal saponins).

In certain embodiments, the first composition of the invention comprises at least about 0.001%, 0.01%, 0.1%, 1%, 2%, 3%, 4%, 5%, 6%, 7%, 8%, 9%, 10%, 15%, 20%, 30%, 40%, 50%, 60%, 70%, 80%, 90%, 95%, or at least about 99% w/w (by weight of the final composition w/w) of saponin(s), such as quillaja and /or Yucca saponin(s).

For example, the first composition of the invention comprises (or consist essentially/consist of) i) at least one Lamiaceae extract (such as rosemary and / or salvia extract) comprising carnosol and /or carnosic acid and ii) at least one saponin, (such as quillaja and/or yucca saponin(s))
wherein the at least one saponin is at a concentration of at least about 0.001%, 0.01%, 0.1%, 1%, 2%, 3%, 4%, 5%, 6%, 7%, 8%, 9%, 10%, 15%, 20%, 30%, 40%, 50%, 60% % w/w (by weight of the final composition w/w), such as at least 4%, such as at least 10%,
, and wherein the carnosic acid concentration is from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w) of carnosic acid, such as from about 20% to about 80% w/w, preferably such as from about 30% to about 50 % w/w, such as about 40% w/w; and/ or
the wherein the carnosol concentration is from about 1%, 5%, 10%, 15%, 20%, 25%, 30%, 35%, 40% or 45% to about 95%, 90%, 85%, 80%, 75%, 70%, 65%, 60%, 55% or 50% w/w (by weight of the final composition w/w), such as from about 1% to 20% w/w, such as from about 2 % to about 10% w/w, preferably such as from about 3 % to about 5% w/w, such as about 4% w/w.

In certain embodiments the ratio between the at least one Lamiaceae extract (such as rosemary and / or salvia extract) and the at least one saponin (such as quillaja and/or yucca saponin(s)) is from about 30:1 to 1:30, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is from about 5:1 to about 1:5, such as about 1:1 or 1.5:1.

In certain embodiments, the at least one Lamiaceae extract (such as rosemary and / or salvia extract) comprises phenolic diterpen(s) and the ration between said phenolic diterpen(s) and the at least one saponin (such as quillaja and/or yucca saponin(s)) is from about 30:1 to 1:30, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is from about 5:1 to about 1:5, such as about 1:1 or 1.5:1.

In another embodiment, the Lamiaceae extract comprise (or consist essentially/consist of) the phenolic diterpens carnosic acid and/or carnosol. In another embodiment, the ratio between carnosic acid and carnosol is from about 40:1 to about 1:40, such as such as 30:1, 20:1, 10:1, 5:1 or 1:1.

In certain embodiments, the at least one saponin comprises (or consist essentially/consist of) quillaja and yucca saponin(s). In a preferred embodiment, the ratio between the quillaja and the yucca saponin(s) is from about 20:1 to 1:20, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is about 3:1.

In a further aspect, the invention is related to a composition (or second composition of the invention) comprising i) at least one phenolic diterpene (such as carnosic acid and /or carnosol) and/or ii) at least one saponin.

Phenolic diterpenes and saponins have been described previously. The details, examples and preferences provided in relation to any one or more of the stated aspects of the first composition related to phenolic diterpens and saponins may be further apply equally to all aspects of the second composition of the invention

In an embodiment of the second composition of the invention, the phenolic diterpene(s) (such as carnosic acid or carnosol) is a compound extracted from nature or synthesized.

In an embodiment of the second composition of the invention the carnosic acid is a “Pure carnosic acid extract”. This means a carnosic acid extract that has a concentration of at least 80% of carnosic acid, such as 95%

In an embodiment of the second composition of the invention the carnosol is a “Pure carnosol extract”. This means a carnosol extract that has a concentration of at least 80% of carnosol, such as 95%.

In a preferred embodiment the phenolic diterpene(s) (such as carnosic acid or carnosol) is of natural origin (such as from a Lamiaceae extract from, for example, rosemary and/or salvia).

In certain embodiments, the concentration of phenolic diterpene(s) in the final composition is: from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w), such as from about 20% to about 80% w/w, preferably such as from about 30% to about 70 % w/w such as about 50% w/w.

In a preferred embodiment the phenolic diterpen(s) are carnosic acid and/or carnosol. In certain embodiments, the ratio between carnosic acid and carnosol is from 40:1 to 5:1, such as 30:1, 20:1, 10:1 or 5:1.

In another embodiment of the second composition of the invention, the concentration of the following phenolic diterpenes in the final composition is:
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w) of carnosic acid, such as from about 20% to about 80% w/w, preferably such as from about 30% to about 50 % w/w such as about 40% w/w; and/ or
b) from about 1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition(w/w), such as from about 1% to 20% w/w such as from about 2 % to about 10% w/w, preferably such as from about 3 % to about 5% w/w, such as about 4% w/w.

Saponin(s) have been described previously and all the embodiments disclosed and exemplified previously regarding the saponins of the first composition of the invention can be also applied to the second composition of the invention.

For instance, the saponin(s) of the second composition of the invention may be of natural or synthetic origin.

For example, the at least one saponin (such as a purified saponin(s) or a saponin extract) is obtained or obtainable from Quillaja (such as Q. saponaria) and from Yucca (such as Yucca schidigera).

For example, the second composition of the invention comprises (or consist essentially/consist of) i) at least one phenolic diterpen (such as carnosic acid and / or carnosol) and/or ii) at least one saponin (such as quillaja and/or yucca saponin(s))
wherein the at least one saponin is at a concentration of at least about 0.001%, 0.01%, 0.1%, 1%, 2%, 3%, 4%, 5%, 6%, 7%, 8%, 9%, 10%, 15%, 20%, 30%, 40%, 50%, 60% % w/w (by weight of the final composition w/w), such as at least 4%, such as at least 10% w/w,
and wherein the carnosic acid concentration is from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w) of carnosic acid, such as from about 20% to about 80% w/w, preferably such as from about 30% to about 50 % w/w, such as about 40% w/w; and/ or
the carnosol concentration is from about 1%, 5%, 10%, 15%, 20%, 25%, 30%, 35%, 40% or 45% to about 95%, 90%, 85%, 80%, 75%, 70%, 65%, 60%, 55% or 50% w/w (by weight of the final composition w/w), such as from about 1% to 20% w/w, such as from about 2% to about 10% w/w, preferably such as from about 3% to about 5% w/w, such as about 4% w/w.

In certain embodiments of the second composition of the invention, the ratio between the phenolic diterpen (such as carnosic acid and / or carnosol) and the at least one saponin is from 30:1 to 1:30, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is from about 5:1 to about 1:5, such as about 1:1 or about 1.5:1.

In a preferred embodiment, the at least one phenolic diterpene is selected from carnosic acid, carnosol or a mixture thereof. In certain embodiments, the ratio between carnosic acid and carnosol is from 40:1 to 5:1, such as 30:1, 20:1, 10:1 or 5:1.

In certain embodiments, the at least one saponin comprises (or consist essentially/consist of) quillaja and yucca saponin(s). In a preferred embodiment, the ratio between the quillaja and the yucca saponin is from about 20:1 to 1:20, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is about 3:1.

The invention also relates to a composition (or third composition of the invention) comprising at least one saponin (such as a quillaja and/or yucca saponin(s)) and a citrus juice (such as lemon juice) and/or hesperidin

Citrus juice (such as lemon, orange, bitter orange, mandarin juice,) may be produced produced by vacuum drying. For example one kg of the product may be made from around 12.4 kg single strength juice at 8°Brix, obtained from around 21.5 kg of fresh fruit. Citrus juice powder contains fruit solids and any anticaking agent (such as silicon dioxide).

Hesperidin is a bioflavonoid found primarily in citrus fruit such as oranges, grapefruit, lemon, tangerines etc.

Hesperidin may be extracted by any method known in the art. For example dried immature fruits (citrus aurantium L.) are exposed to a vapor in order to remove pectins prior to the extraction with water. Subsequently, sodium hydroxide and calcium hydroxide may be added in the solution to stabilize the pH value. Filtration steps may be also used. Following the filtration step, an acidification of the filtrate may be used for example by using a strong or weak acid (for example using using HCI). Upon this step the hesperidin precipitates, the liquid solution is removed and the precipitate is dried.

“Hesperidin” means a compound extracted from nature or synthesized.

The “Hesperidin” contains at least 50%, such as 60%, such as at least 70%, 80%, 85%, 90% or at least 99% hesperidin, preferably more than 95% of hesperidin as measured by HPLC.

In certain embodiments, the hesperidin is “Pure hesperidin extract”.

“Pure hesperidin extract” means a hesperidin extract that has a concentration of at least 80% hesperidin.

In one embodiment, the saponin is a yucca extract with about 9% of saponins, the citrus juice is a lemon dry juice with and the hesperidin is a The ratio of saponis/citrus juice and hesperidin is extracted from Citrus aurantium (at least 80% hesperidin) and the ratio is about 1.5:2:1.5, or about 1:1:1, or about 1:1:2, or about 2:2:1, or about 2:1:2, or about 1:2:1.

In certain embodiments of the third composition of the invention, the ratio between the saponin and hesperidin is from about 0.1:1 to 1:1, such as 0.3:1, 0.5:1, or 0.6:1.

The first, second or third composition of the invention may optionally comprise a plant and/or vegetable oil. For example, the composition(s) of the invention may comprise plant and/or vegetable oils selected from the group consisting of coconut oil, corn oil, cottonseed oil, olive oil, palm oil, peanut oil (ground nut oil), rapeseed oil, including canola oil, safflower oil, sesame oil, soybean oil, sunflower oil, and mixtures thereof.

The composition(s) of the invention may be provided in a solid or liquid form, preferably a solid form, such as a powder. By solid form, it is included that the compound may be provided as an amorphous solid, or as a crystalline or part-crystalline solid.

The present invention also relates to a food or beverage product for humans or animals, a nutritional supplement, a nutraceutical formulation, a fragrance or flavouring, a pharmaceutical or veterinary formulation, an oenological or cosmetic formulation (from now on “a product”) comprising a first composition,a second composition and/or a third composition of the invention. In certain embodiment, the food product is selected from beverages such as carbonated and non carbonated beverages (such as fruit drinks, teas, coffees, enhanced waters etc) and emulsified or non-emulsified sauces (such as mayonnaise, ketchup, etc)

Food encompasses the following general food categories, as defined by the Food and Drug Administration (FDA): baked goods and baking mixes, including all ready-to-eat and ready-to-bake products, flours, and mixes requiring preparation before serving; beverages, alcoholic, including malt beverages, wines, distilled liquors, and cocktail mix; beverages and beverage bases, non-alcoholic, including only special or spiced teas, soft drinks, coffee substitutes, and fruit and vegetable flavored gelatin drinks; breakfast cereals, including ready-to-eat and instant and regular hot cereals; cheeses, including curd and whey cheeses, cream, natural, grating, processed, spread, dip, and miscellaneous cheeses; chewing gum, including all forms; coffee and tea, including regular, decaffeinated, and instant types; condiments and relishes, including plain seasoning sauces and spreads, olives, pickles, and relishes, but not spices or herbs; confections and frostings, including candy and flavored frosting, marshmallows, baking chocolate, and brown, lump, rock, maple, powdered, and raw sugars; dairy product analogs, including nondairy milk, frozen or liquid creamers, coffee whiteners, toppings, and other nondairy products; egg products, including liquid, frozen, or dried eggs, and egg dishes made therefrom, i.e., egg roll, egg foo young, egg salad, and frozen multicourse egg meals, but not fresh eggs; fats and oils, including margarine, dressings for salads, butter, salad oils, shortenings and cooking oils; fish products, including all prepared main dishes, salads, appetizers, frozen multicourse meals, and spreads containing fish, shellfish, and other aquatic animals, but not fresh fish; fresh eggs, including cooked eggs and egg dishes made only from fresh shell eggs; fresh fish, including only fresh and frozen fish, shellfish, and other aquatic animals; fresh fruits and fruit juices, including only raw fruits, citrus, melons, and berries, and home-prepared “ades” and punches made therefrom; fresh meats, including only fresh or home-frozen beef or veal, pork, lamb or mutton and home-prepared fresh meat-containing dishes, salads, appetizers, or sandwich spreads made therefrom; fresh poultry, including only fresh or home-frozen poultry and game birds and home-prepared fresh poultry-containing dishes, salads, appetizers, or sandwich spreads made therefrom; fresh vegetables, tomatoes, and potatoes, including only fresh and home-prepared vegetables; frozen dairy desserts and mixes, including ice cream, ice milks, sherbets, and other frozen dairy desserts and specialties; fruit and water ices, including all frozen fruit and water ices; gelatins, puddings, and fillings, including flavored gelatin desserts, puddings, custards, parfaits, pie fillings, and gelatin base salads; grain products and pastas, including macaroni and noodle products, rice dishes, and frozen multicourse meals, without meat or vegetables; gravies and sauces, including all meat sauces and gravies, and tomato, milk, buttery, and specialty sauces; hard candy and cough drops, including all hard type candies; herbs, seeds, spices, seasonings, blends, extracts, and flavorings, including all natural and artificial spices, blends, and flavors; jams and jellies, home-prepared, including only home-prepared jams, jellies, fruit butters, preserves, and sweet spreads; jams and jellies, commercial, including only commercially processed jams, jellies, fruit butters, preserves, and sweet spreads; meat products, including all meats and meat containing dishes, salads, appetizers, frozen multicourse meat meals, and sandwich ingredients prepared by commercial processing or using commercially processed meats with home preparation; milk, whole and skim, including only whole, lowfat, and skim fluid milks; milk products, including flavored milks and milk drinks, dry milks, toppings, snack dips, spreads, weight control milk beverages, and other milk origin products; nuts and nut products, including whole or shelled tree nuts, peanuts, coconut, and nut and peanut spreads; plant protein products, including the National Academy of Sciences/National Research Council “reconstituted vegetable protein” category, and meat, poultry, and fish substitutes, analogues, and extender products made from plant proteins; poultry products, including all poultry and poultry-containing dishes, salads, appetizers, frozen multicourse poultry meals, and sandwich ingredients prepared by commercial processing or using commercially processed poultry with home preparation; processed fruits and fruit juices, including all commercially processed fruits, citrus, berries, and mixtures; salads, juices and juice punches, concentrates, dilution, “ades”, and drink substitutes made therefrom; processed vegetables and vegetable juices, including all commercially processed vegetables, vegetable dishes, frozen multicourse vegetable meals, and vegetable juices and blends; snack foods, including chips, pretzels, and other novelty snacks; soft candy, including candy bars, chocolates, fudge, mints, and other chewy or nougat candies; soups, home-prepared, including meat, fish, poultry, vegetable, and combination home-prepared soups; soups and soup mixes, including commercially prepared meat, fish, poultry, vegetable, and combination soups and soup mixes; sugar, white, granulated, including only white granulated sugar; sugar substitutes, including granulated, liquid, and tablet sugar substitutes; and sweet sauces, toppings, and syrups, including chocolate, berry, fruit, corn syrup, and maple sweet sauces and toppings.

The food or beverage product for humans or animals, a nutritional supplement, a nutraceutical formulation, a fragrance or flavouring, a pharmaceutical or veterinary formulation, an oenological or cosmetic formulation comprises from about 0.0001 %, about 0.001%, about 0.01, about 0.1, about 1, 1.5, 2, 2.5, 3, 3.5, 4, 4.5, 5 or 5.5, to about 20%, about 10, about 9, 8, 7, or about 6% w/w of the first composition,a second composition and/or a third composition of the invention. In certain embodiments, the food or beverage product for humans or animals, a nutritional supplement, a nutraceutical formulation, a fragrance or flavoring, a pharmaceutical or veterinary formulation, an oenological or cosmetic formulationcomprising a first composition of the invention, the concentration of the at least one Lamiaceae extract (such as a rosemary and/or salvia extract) in the final product is from about 0.0001 %, about 0.001%, about 0.01%, about 0.1%, about 1%, 1.5%, 2%, 2%, 2.5%, 3%, 3.5%, 4%, 4.5% or 5%, to about 20%, about 10%, about 9%, 8%, 7%, or about 6% w/w.

In certain embodiments, the Lamiaceae extract(s) (such as a rosemary and/or salvia extract) comprises (or consist essentially/consist of) at least one phenolic diterpen (such as carnosic acid and / or carnosol). In certain embodiments of the product (food or beverage etc), comprising a first composition of the invention, the concentration of carnosic acid in the final product is from about 12 ppm, 15 ppm, 20 ppm, 30, 40, 50, 60, 70, 80, 90, 100 ppm, to about 6%, 5%, 4%, 4%, 3%, 2%, 1% (10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as from 50 ppm to 1000 ppm; and /or the concentration of carnosol in the final product is from about 5 ppm, 10 ppm, 15 ppm, 20 ppm, 30, 40, 50, 60, 70, 80, 90, 100 pp, to about 6%, 5%, 4%, 4%, 3%, 2%, 1% (10000 ppm), 9000 ppm, 8000 ppm, 7000 ppm, 6000 ppm, 5000 ppm, 4000 ppm, 3000 ppm, 2000 ppm,1000 ppm, 800 ppm, 600 ppm, 500 ppm, 400 ppm, 300 ppm to 200 ppm w/w, such as 5 ppm to 100 ppm. In certain embodiments, the ratio between carnosic acid and carnosol is from 40:1 to 5:1, such as 30:1, 20:1, 10:1 or 5:1.

In certain embodiments, the food or beverage product for humans or animals, a nutritional supplement, a nutraceutical formulation, a fragrance or flavouring, a pharmaceutical or veterinary formulation, an oenological or cosmetic formulation comprising a second composition of the invention, has a concentration of the at least one phenolic diterpen (such as carnosic acid and / or carnosol) in the final product from about 12 ppm, 15 ppm, 20 ppm, 30, 40, 50, 60, 70, 80, 90, 100 ppm, to about to about 40%, 30%, 20%, 10%, 9%, 8%, 7%, 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm, such as from 50 ppm to 1000 ppm.

In certain embodiments of the product (such as food or beverage etc), comprising a second composition of the invention, the concentration of carnosic acid in the final product is from about 12 ppm, 15 ppm, 20 ppm, 30, 40, 50, 60, 70, 80, 90, 100 ppm, to about 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as from 50 ppm to 1000 ppm; and /or the concentration of carnosol in the final product is of about 5 ppm, 10 ppm, 15 ppm, 20 ppm, 30, 40, 50, 60, 70, 80, 90, 100 pp, to about 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as 5 ppm to 100 ppm.

In certain embodiments, the ratio between carnosic acid and carnosol is from 40:1 to 5:1, such as 30:1, 20:1, 10:1 or 5:1.

In certain embodiments, the product (food or beverage product for humans or animals, a nutritional supplement, a nutraceutical formulation, a fragrance or flavouring, a pharmaceutical or veterinary formulation, an oenological or cosmetic formulation) comprising a first composition,a second composition and/or a third composition of the invention has a concentration of saponin(s) (such as quillaja and/or yucca saponin(s)) in the final product from about 12 ppm, 15 ppm, 20 ppm, 30, 40, 50, 60, 70, 80, 90, 100 ppm, to about 40%, 30%, 20%, 10%, 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as from about 50 ppm to about 2% w/w (or 20000 ppm). In a preferred embodiment the saponin(s) is a quillaja and a yucca saponin(s) and the ratio between the quillaja and the yucca saponin is from about 20:1 to 1:20, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is about 3:1. In certain embodiments, the product (food or beverage product for humans or animals, a nutritional supplement, a nutraceutical formulation, a fragrance or flavouring, a pharmaceutical or veterinary formulation, an oenological or cosmetic formulation) comprising a third composition of the invention has a concentration of hesperidin (such as pure citrus hesperidin) in the final product from about 12 ppm, 15 ppm, 20 ppm, 30, 40, 50, 60, 70, 80, 90, 100 ppm, to about 40%, 30%, 20%, 10%, 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as from about 50 ppm to about 2% w/w (or 20000 ppm).

### Antimicrobial and Preservative Uses

As demonstrated in examples 2 and 3 and table 17, 18 and table 20, the saponin(s) (such as quillaja and yucca saponins) can inhibit the growth of different spoiler bacteria. Thus the present invention is also related to the use of at least one saponin as antimicrobial and/or as a food preservative.

As demonstrated in examples 1 to 6, the saponin(s) combined with Lamiaceace extracts rich in phenolic diterpens have a synergistic effect and can inhibit the growth of a high spectrum of different spoiler bacteria, yeast and molds. Said synergistic effect can be for example observed on Example 2. On table 9 it can be observed that when applied alone quillaja extracts only used at high concentrations had an effect inhibiting Saccharomyces pombe growth. Only very high concentration in rosemary extract that provide between 0.28% of carnosic acid could inhibit the yeast growth. Surprisingly when used together, in combination, rosemary and quillaja extract fully inhibited Saccharomyces pombe, even a very low concentrations of carnosic acid (0.014%) and quillaja (0.0085%). This is synergy.

Thus the present invention is also related to the use of a first composition and/or the second compositions of the invention as antimicrobial and/or as a food preservative.

The invention also relates to the use of a composition (third composition of the invention) comprising at least one saponin (such as a quillaja and/or yucca saponin(s)), a citrus juice (such as lemon juice) and/or hesperidin as food preservative and/or antimicrobial.

According to the present invention, the antimicrobial properties of the first compositions or the second compositions of the invention are harnessed as a preservative in compositions such as beverages and food, cosmetic formulation etc, by using an effective amount of the first compositions or the second compositions of the invention, wherein the preservative achieves microbial stability of at least one microorganism chosen from mold, yeast and bacteria in said beverage, food or cosmetic formulation.

As used herein, the term “antimicrobial” may refer to a substance (such as a first, second or third composition of the invention) that kills microorganisms or stops (inhibits) their growth. The microorganism may be bacteria, fungi, yeast or a mixture thereof. Methods for assessing the activity of a compound as antimicrobial, such as the methods described in the examples of the present application, are well known by a person skilled in the art.

As used herein, the term “preservative” may refer to substance or a chemical (such as a first or second composition of the invention) that is added to products such as food products, beverages, pharmaceutical drugs, paints, biological samples, cosmetics, and many other products to prevent decomposition by microbial growth or by undesirable chemical changes.

In certain embodiments, the preservative and/or antimicrobial effect is against spoilage microorganism (such as food and/or beverage spoilage microorganism).

As used herein, the term “Spoilage” refers to the consequence of the microorganism (bacteria yeast and/or mold) growth in the product such as a food or beverage product for humans or animals, a nutritional supplement, nutraceutical formulation, a dietary, a sports supplement, a fragrance or flavouring, a pharmaceutical or veterinary formulation, an oenological or cosmetic formulation. The product components (such as food, beverage or cosmetic formulation components) are utilized as growth substrates by the microorganism (such as bacteria, yeast and /or mold) and are transformed into a vast array of metabolic end products. In this way, the chemical, physical, and sensory properties of the product (such as food, beverage, cosmetic formulation etc) food are changed. Microorganism spoilage becomes evident to the consumer in many ways, depending on the product: signs of food spoilage may include an appearance different from the food in its fresh form, such as a change in color, a change in texture, an unpleasant odour, or an undesirable taste. The item may become softer than normal. If mold occurs, it is often visible externally on the item. As used herein, the term “food and beverage spoilage microorganism” (such as food and beverage spoilage bacteria, food and beverage spoilage yeast and food and beverage spoilage molds) refers to a microorganism that growths in said food or beverage product and /or can be isolated from said spoiled product and changes the chemical, physical, and/or sensory properties of the food or beverage such a change in colour, a change in texture, an unpleasant odour and / or an undesirable taste. Spoilage microorganism can also produce toxins and other substances that are dangerous to the consumers.

In certain embodiments the preservative and/or antimicrobial effect is against bacteria, more precisely food and beverage spoilage bacterial. In certain embodiments the antibacterial effect is against vegetative cells. In certain embodiments the antibacterial effect is against the spores of the bacterial.

The antibacterial effect may be against acid-lactic bacteria, thermo-acidophilic bacteria, xerophilic bacteria and/or acidophilic bacteria.

As used herein, the term “Acid lactic bacteria” refers to an order of gram-positive, low-GC, acid-tolerant, generally nonsporulating, non-respiring, either rod-shaped (bacilli) or spherical (cocci) bacteria that share common metabolic and physiological characteristics. These bacteria produce lactic acid as the major metabolic end product of carbohydrate fermentation. Examples of acid lactic bacteria are the Lactobacillus genus (such as Lactobacillis plantarum), Leuconostoc genus or Lactococcus genus.

As used herein, the term “Thermo-acidophilic” refers to an extremophilic microorganism that is both thermophilic and acidophilic; i.e., it can grow under conditions of high temperature and low pH. Examples of Thermo-acidophilic bacteria are bacteria from the Alicyclobacillus genus such as Alicyclobacillus acidoterrestris and Alicyclobacillus acidocaldarius. These types of bacteria are able to grow in acidic conditions, while the spores are able to survive typical pasteurization procedures.

As used herein, the term “Xerophilic bacteria” refers to bacteria that are tolerant to high osmotic pressure.

As used herein, the term “Acidophilic bacteria” refers to bacteria that thrive under acidic and high acidic conditions. Examples are Alicyclobacillus genus (such as Alicyclobacillus acidoterrestris or Alicyclobacillus acidocaldarius ), Gluconobacter genus and Zymomonas genus.

The anti-bacteria effect (bacterial growth inhibited decreases and/or limited) may be against bacteria that belong to the genus and/or species mentioned before or combination thereof.

In another embodiment, the preservative and/or antimicrobial effect is against yeast, more precisely food and beverage spoilage yeast. Typically, spoilage by yeasts manifests itself as fermentation with gas and ethanol production, as well as sedimentation, off-flavours and odours, and loss of cloud or emulsion stability. Examples of yeast spoilage include “blown cans” of soft drinks, cloudy re-fermented wine, pink or red slime dripping from refrigerated meat, white yeast colonies on food, and tainted fruit juices.

Yeasts are eukaryotic, single-celled microorganisms classified as members of the fungus kingdom. In certain embodiments, the yeast is able to degrade glucose or fructose to ethanol, even in the presence of oxygen (Crabtree effect) (such as Saccharomyces cerevisiae or Dekkera anomala), is tolerant to high osmotic pressure (xerophilic) (such as Schizosaccharomyces pombe) and /or is tolerate high ethanol concentrations (such as Dekkera anomala).

Common genera (or genus) of yeast include: Candida Spp, Saccharomyces Spp. (such as Saccharomices cerevisiae and/or Saccharomyces pombe), Dekkera Spp. (such as D. anomala), Brettanomyces Spp. and/or Zygosaccharomyces Spp (such as Zygosaccharomyces baillii).

The anti-yeast effect (yeast growth inhibited, decreases and/or limited) may be against yeast that belongs to the genus and/or species mentioned before or combination thereof.

In another embodiment, the preservative and/or antimicrobial effect is against molds. As use herein the term “mold” or “mould” refers to a fungus that grows in the form of multicellular filaments called hyphae. Molds are a large and taxonomically diverse number of fungal species in which the growth of hyphae results in discoloration and a fuzzy appearance, especially on food. Molds are considered to be microbes and do not form a specific taxonomic or phylogenetic grouping, but can be found in the divisions Zygomycota and Ascomycota. Common genera of molds include:

Acremonium, Alternaria, Aspergillus, Byssochlamys, Cladosporium, Fusarium, Mucor, Penicillium, Rhizopus, Stachybotrys, Trichoderma and Trichophyton. More precisely food and beverage spoilage molds such as the molds selected from Byssochlamys genus (such as Byssochlamys nivea), Penicillium genum and Aspergillus genum (such as Aspergillum niger). Aspergillum niger is a co-nidial fungus with an asexual spore-forming structure that is highly thermotolerant, adaptive UV radiations and able to grow at low aw food products (growth at aw > 0.80) and within a wide range of pH (2 - 8). It is also producer of mycotoxin and therefore a dangerous food and beverage spoiler.

The anti-mold effect (mold growth inhibited, decreases and/or limited) may be against molds that belong to the genus and/or species mentioned before or combination thereof.

The invention also relates to the use of a composition comprising at least one saponin (such as a quillaja and/or yucca saponin(s)) as food preservative and/or antimicrobial. As demonstrated in table 16, 17, 18 and 20 the saponin(s) can inhibit the growth of different microorganism.

In certain embodiments, the saponin is from yucca and the microorganism(s) are selected from bacteria, yeast and/or mold. In a preferred embodiment the microorganisms is selected from Lactobacillus genus (such as Lactobacillus plantarum), Alicyclobacillus genus (such as Alicyclobacillus acidoterrestris), Saccharomyces genus such as (Saccharomyces cerevisiae, Saccharomyces pombe) and / or Zygosaccharomyces (such as Zygosaccha-romyces baillii)

The invention also relates to method for processing food comprising applying to or incorporating into a food, a composition comprising at least one saponin (such as a quillaja and/or yucca saponin) for having a food preservative and/or antimicrobial effect.

The invention also relates to method for processing a food comprising applying to or incorporating into a food, a first, a second and/or a third composition of the invention for having a food preservative and/or antimicrobial effect.

The inventors of the present invention have also surprisingly found that the combination of at least one Lamiaceae extracts and at least one saponin (such as quillaja and/or yucca saponin(s)) has an antimicrobial synergistic effect (a synergistic anti-yeast, anti mold and/or antibacterial effect)

This can be observed on examples 1 to 6.

The inventors have also shown that the combination of quillaja and yucca saponins with Lamiaceae extracts shown synergistic antimicrobial effect even against very resistant yeast strains such as Dekkera anomala (examples 5 and 6)

The invention also relates to preservative and/or antimicrobial composition (such as an antibacterial, anti-yeast or anti-mold) comprising an effective amount of a first composition of the invention, an effective amount of a second composition of the invention and/or an effective amount of the third composition of the invention.

“Effective amount” is the amount necessary in order to achieve a specific effect, in accordance with what one of ordinary skill in the art would be readily able to determine through routine experimentation. For example, with regard to the present disclosure, an effective amount of a composition comprising a Lamiaceae extract and at least one saponin to be applied to a food product or foodstuff, e.g. meat, fish and poultry (both fresh/unprocessed and processed) or beverage, to extend the longevity of the food, e.g. red color to the fresh meat, fish and poultry, is an amount which is determined to provide the red color longevity based on known parameters which include, but are not limited to the concentration of the saponin(s) and a Lamiaceae extract, the volume and/or surface area of the meat, fish and poultry, and the atmospheric environment conditions of the meat, fish and poultry. Similarly, the effective amounts of the phenolic diterpenes and the at least one saponin(s) to extend the longevity of red color to the meat, fish and poultry are determined in a similar way.

The effective amount of the saponin(s) (such as a quillaja and/or yucca saponin(s) comprising extract) can depend on the nature of the food product or beverage. For example, the saponin(s) (like saponin(s) comprising extract from quillaja and/or yucca) may be present in the food product, such as a beverage product, in an amount ranging from about 50 ppm to about 20,000 ppm such as from about 250 ppm to about 5000 ppm in high-nutrient food product (e.g., juice, vitamin, nitrogen, etc. beverages), or for example from about 100 ppm to about 1000 ppm in low nutrient food (e.g., beverages lacking vitamin(s), low levels of nitrogen, etc., and about < 3% juice), and further for example, from about 250 ppm to about 1000 ppm such as 250 ppm to 750 ppm in low acid food product (such as low acid beverages)

The phenolic diterpene(s) selected from carnosic acid, carnosol, and mixtures thereof may be present in the food, such as a beverage product in an amount ranging from about 50 ppm to about 20,000 ppm such as from about 250 ppm to about 5000 ppm in high-nutrient food product (e.g., juice, vitamin, nitrogen, etc. beverages), or for example from about 100 ppm to about 1000 ppm in low nutrient food (e.g., beverages lacking vitamin(s), low levels of nitrogen, etc., and about < 3% juice) and further for example, from about 250 ppm to about 1000 ppm such as 250 ppm to 750 ppm in low acid food product (such as low acid beverages)

The invention also relates to the use of a first composition of the invention and/or of a second composition of the invention as antimicrobial and/or preservative in a food or beverage product for humans or animals, a nutritional supplement, a nutraceutical formulation, a fragrance or flavoring, a pharmaceutical or veterinary formulation, an oenological or cosmetic formulation.

The present invention also provides a process for producing a food or beverage product for humans or animals, a nutritional supplement, a nutraceutical formulation, a fragrance or flavouring, a pharmaceutical or veterinary formulation, an oenological or cosmetic formulation comprising supplementing (or adding to) a food or beverage product for humans or animals, a nutritional supplement, a nutraceutical formulation, a fragrance or flavouring, a pharmaceutical or veterinary formulation, an oenological or cosmetic formulation with a first a second and/or a third composition of the invention.

When used as an antimicrobial and/or preservative of food or beverage product for humans or animals, a nutritional supplement, a nutraceutical formulation, a fragrance or flavouring, a pharmaceutical or veterinary formulation, an oenological or cosmetic formulation, an effective amount of the first and/or the second composition of the invention may be added at any appropriate step in the production process of said food or beverage product, etc, to achieve the desired effect (i.e antimicrobial and/or preservative).

The invention also relates to a method for processing or preparing the above mentioned products (such a food or beverage) comprising applying to or incorporating into said product (such as a food or beverage), a first a second and/or a third composition of the invention for having a food preservative and/or antimicrobial effect.

The present invention is also related to a method for processing or preparing the above mentioned products (such a food or beverage) comprising applying to or incorporating into said product (such as a food or beverage), a first composition of the invention comprising at least one saponin (such as a quillaja and/or yucca saponin(s)) and at least one Lamiaceae extract such as a rosemary extract and/or salvia extract. In certain embodiments, Lamiaceae extract comprises at least one phenolic diterpene selected from carnosic acid, carnosol, and mixtures thereof.

The present invention also related to a method for processing or preparing the above mentioned products (such a food or beverage) comprising applying to or incorporating into said product (such as a food or beverage), a second composition of the invention comprising at least one saponin (such as a quillaja and/or yucca saponin) and at least one phenolic diterpene selected from carnosic acid, carnosol, and mixtures thereof.

In certain embodiments the food is selected from meat, poultry, fish; dairy products (milk, butter, cheese); beverage products; bakery products; canned foods; fruit and confectionery products; and emulsified or note emulsified sauces (such as mayonnaise, ketchup, etc)

In a preferred embodiment, the method extends the colour life, inhibits, decreases and/or limits microbial growth (such as bacterial, yeast and/or molds growth) in said product (such as a food or beverage). The antibacterial effect may be against acid-lactic bacteria, thermo-acidophilic bacteria (such as Lactobacillus genus (such as Lactobacillis plantarum), Leuconostoc genus and/or Lactococcus genus), Xerophilic bacteria (such as Alicyclobacillus genus like Alicyclobacillus acidoterrestris or Alicyclobacillus acidocal-darius) and/or acidophilic bacteria (such as Alicyclobacillus genus (such as Alicyclobacillus acidoterrestris or Alicyclobacillus acidocaldarius ), Gluconobacter genus and/or Zymomonas genus), or combinations thereof.

In certain embodiments, the preservative and/or antimicrobial effect is against yeast, more precisely food and beverage spoilage yeast. In certain embodiments, the yeast is selected from the genus Candida Spp, Saccharomyces Spp. (such as Saccharomices cerevisiae and/or Saccharomyces pombe), Dekkera Spp. (such as D. anomala), Brettanomyces spp. and/or Zygosaccharomyces Spp (such as Zygosaccharomyces baillii). The anti-yeast effect may be against one or more yeast, such as the yeast that belongs to the genus and/or species mentioned before or combinations thereof.

In certain embodiments, the preservative and/or antimicrobial effect is against molds such as Acremonium, Alternaria, Aspergillus, Byssochlamys, Cladosporium, Fusarium, Mucor, Penicillium, Rhizopus, Stachybotrys, Trichoderma and Trichophyton. More precisely food and beverage spoilage molds such as the molds selected from Byssochlamys genus (such as Byssochlamys nivea), Penicillium genum and Aspergillus genum such as Aspergillum niger. The anti-mold effect may be against one or more molds, such as the molds that belong to the genus and/or species mentioned before or combinations thereof.

The antimicrobial effect may be against one or more of the bacteria, yeast and molds described before.

In certain embodiments, the method prevents a change in colour, a change in texture, an unpleasant odour and / or an un-desirable taste.

In certain embodiments of the uses or methods described before, the fist composition of the invention comprises (or consist essentially/consist of) a Salvia extract (such as Salvia apiana and Salvia officinalis). In a preferred embodiment, the Salvia extract is enriched in phenolic diterpenes. In certain embodiment, when the composition consist of an extract from Salvia, the antimicrobial effect may be against the following microorganisms: Lactobacillus genus (such as Lactobacillus plantarum), Alicyclobacillus genus (such as Alicyclobacillus acidoterrestris), Saccharomyces genus (such as Saccharomyces cerevisiae or Saccharomyces pombe), Dekkera genus (such as Dekkera anomala), and combinations thereof.

The different saponins and phenolic diterpens were described before.

In certain embodiments the saponin(s) present in the first, second or third composition of the invention are quaillaja saponin(s) and the microorganism are selected from one or more bacteria, yeast and/or mold. The bacteria may be selected from: acid-lactic bacteria, thermo-acidophilic bacteria, Xerophilic bacteria and/or acidophilic bacteria. In a preferred embodiment the bacteria are selected from the Lactobacillus genus (such as Lactobacilus plantarum), the Alicyclobacillus genus (such as Alicylobacilus acidoterrestris) or combinations thereof. In another embodiment the yeast are selected from yeast that are able to: degrade glucose or fructose to ethanol, even in the presence of oxygen (Crabtree effect) (such as Saccharomyces cerevisiae or Dekkera anomala), a yeast that is tolerant to high osmotic pressure (xerophilic) (such as Schizosaccharomyces pombe) and /or a yeast that tolerate high ethanol concentrations (such as Dekkera anomala) or combinations thereof. In another embodiment the yeast is selected from the genus Saccharomyces (such as Saccharomyces pombe and/or Saccaromyces cerevisae), from the genus Dekkera (such as Dekkera anomala) or combinations thereof.

In certain embodiments the saponin(s) present in the first, second or third composition of the invention are yucca saponin(s) and the microorganisms are selected from bacteria, yeast and/or mold or combinations thereof. The bacteria may be selected from: acid-lactic bacteria, thermo-acidophilic bacteria, Xerophilic bacteria and/or acidophilic bacteria. In a preferred embodiment the bacteria are selected from the Lactobacillus genus (such as Lactobacilus plantarum), the Alicyclobacillus genus (such as Alicylobacilus acidoterrestris). In another embodiment the microorganisms are selected from one or more yeast such as yeast that are able to degrade glucose or fructose to ethanol, even in the presence of oxygen (Crabtree effect) (such as Saccharomyces cerevisiae or Dekkera anomala), a yeast that is tolerant to high osmotic pressure (xerophilic) (such as Schizosaccharomyces pombe) and /or is tolerate high ethanol concentrations (such as Dekkera anomala) or combinations thereof. In another embodiment the yeast is selected from the genus Saccharomyces (such as Saccharomyces pombe and/or Saccaromyces cerevisae), from the genus Dekkera (such as Dekkera anomala) or combinations thereof. The molds can be selected from the genus Byssochlamys (such as Byssochlamys nivea).

In one preferred embodiment, the saponin(s) present in the first, second or third composition of the invention are yucca saponin(s) and the microorganisms are selected from the bacteria from the Lactobacillus genus (such as Lactobacilus plantarum (such as Dekkera anomala), the molds from the genus Byssochlamys (such as Byssochlamys nivea) or combinations thereof.

In certain embodiments the saponin(s) present in the first, second or third composition of the invention are from yucca and quillaja and the microorganisms are one or more yeast from the genus Dekkera (such as Dekkera anomala).

The invention is also related to the product (food or beverage product for humans or animals, a nutritional supplement, a nutraceutical formulation, a fragrance or flavouring, a pharmaceutical or veterinary formulation, an oenological or cosmetic formulation) obtained by the above mentioned method(s) for processing. In a preferred embodiment, the product is food or beverage.

In an embodiment, the product (such as food or beverage) product obtained by the above mentioned method(s) has from about 0.0001 %, about 0.001%, about 0.01%, about 0.1%, about 1%, 1.5%, 2%, 2%, 2.5%, 3%, 3.5%, 4%, 4.5% or 5%, to about 20%, about 10%, about 9%, 8%, 7%, or about 6% w/w, such as from 50 ppm to 2% of saponin(s) (such as quillaja and/or yucca saponin(s)) in the final product.

In certain embodiments, the product (such as food or beverage) obtained by the above mentioned method(s) comprising a product (such as food or beverage) and first composition of the invention has from about 0.0001%, about 0.001%, about 0.01%, about 0.1%, about 1%, 1.5%, 2%, 2%, 2.5%, 3%, 3.5%, 4%, 4.5% or 5%, to about 20%, about 10%, about 9%, 8%, 7%, or about 6% w/w of the at least one Lamiaceae extract (such as a rosemary and/or salvia extract) in the final product. In certain embodiments, the product (such as food or beverage) obtained by the above mentioned method(s) comprising a product (such as food or beverage) and first composition of the invention has from about 0.0001 %, about 0.001%, about 0.01%, about 0.1%, about 1%, 1.5%, 2%, 2%, 2.5%, 3%, 3.5%, 4%, 4.5% or 5%, to about 20%, about 10%, about 9%, 8%, 7%, or about 6% w/w or more of Lamiaceae phenolic diterpene selected from carnosic acid, carnosol, and mixtures thereof, and 0.0001 %, about 0.001%, about 0.01%, about 0.1%, about 1%, 1.5%, 2%, 2%, 2.5%, 3%, 3.5%, 4%, 4.5% or 5%, to about 20%, about 10%, about 9%, 8%, 7%, or about 6% w/w, such as from 50 ppm to 2% or more of saponin(s) (such as quillaja and/or yucca saponin(s)) in the final product.

In certain embodiments, the product (such as food or beverage) obtained by the above mentioned method(s) comprising a product (such as food or beverage) and first or a second composition of the invention has from about 12 ppm, 15 ppm, 20 ppm, 30, 40, 50, 60, 70, 80, 90, 100 ppm, to about 6%, 5%, 4%, 4%, 3%, 2%, 1%(or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as from 50 ppm to 1000 ppm of carnosic acid in the final product ; and /or from about 5 ppm, 10 ppm, 15 ppm, 20 ppm, 30, 40, 50, 60, 70, 80, 90, 100 pp, to about 6%, 5%, 4%, 4%, 3%, 2%, 1%(or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm w/w, such as 5 ppm to 100 ppm of carnosol in the final product. In certain embodiments, the ratio between carnosic acid and carnosol is from 40:1 to 5:1, such as 30:1, 20:1, 10:1 or 5:1.

In an embodiment, the product (such as food or beverage) obtained by the above mentioned method(s) comprising a product (such as food or beverage) and a first and/or a second composition of the invention has from about 12 ppm, 15 ppm, 20 ppm, 30, 40, 50, 60, 70, 80, 90, 100 ppm, to about 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm, such as from 50 ppm to 1000 ppm of carnosic acid in the final product; and /or from about 5 ppm, 10 ppm, 15 ppm, 20 ppm, 30, 40, 50, 60, 70, 80, 90, 100 pp, to about 6%, 5%, 4%, 4%, 3%, 2%, 1% w/w (or 10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm, such as 5 ppm to 100 ppm of carnosol in the final product. In certain embodiments, the ratio between carnosic acid and carnosol is from 40:1 to 5:1, such as 30:1, 20:1, 10:1 or 5:1 or more.

In an embodiment of the used, methods and food products previously mentioned, the first or the second composition may be in the form of a composition which additionally contains a carrier selected from the group consisting of arabic gum, dextrose, salt, mono & diglycerides of fatty acids, MPG, Polysorbate 80, dextrose, vegetable oil, glucose syrup, glycerin, decaglycerol monooleate, fatty acid esters, benzyl alcohol, ethyl alcohol, propylene, glycol, polysorbates, sorbi-tans, sorbitan trioleate, capric/caprylic triglycerides, dextrose and combinations thereof.

In a preferred embodiment, the composition is in dry form. In another embodiment, the composition may be in a liquid form.

In an embodiment of the uses, methods and products (such as food or beverages) described previously, the Lamiaceae extract is essentially free of native essential oil. In an embodiment, of the uses, methods and food products described previously, the majority of volatile oil components have been removed from the Lamiaceae extract.

In an embodiment of the uses, methods and products (such as food or beverages) described previously the ration between the at least one Lamiaceae extract (such as rosemary and / or salvia extract) and the at least one saponin (such as quillaja and/or yucca saponin(s)) is from about 30:1 to 1:30, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is from about 5:1 to about 1:5, such as about 1:1, 1.5:1.

In an embodiment of the uses, methods and products (such as food or beverages) described previously, the ratio between the phenolic diterpen(s) and the at least one saponin (such as quillaja and/or yucca saponin(s)) is from about 30:1 to 1:30, such as 15:1, 10:1, 9:1, 8:1, 7:1, 6:1, 5:1, 4:1, 3:1, 2:1, 1:1, 1:2,1:3, 1:4, 1:4, 1:6, 1:7, 1:8, 1:9, 1:10, 1:15 or about 1:20. In a preferred embodiment the ratio is from about 5:1 to about 1:5, such as about 1:1, 1.5:1.

In certain embodiments the applying to or incorporating into said product (such as a food or beverage), comprise applying the first, the second and/or the third composition, in the form a dry powder or a liquid form, to the food.

In another embodiment, the applying or incorporating into said product (such as food or beverage), comprise applying the components a) and b) of the first, the second and /or the third composition together (as a “ready to use” composition) or separately: for example first component a) and then component b) or first component b) and then component a). In this way the components a) and b) can be added at different points during the production of the product (like a two-step kit composition).

The present invention also relates to a kit comprising the different elements of the first, second and/or third composition of the invention. In certain embodiments, the kit compises the elements already mixed and ready to use (for example as a blend) and optionally instructions of how to use said ingredients.

In certain embodiments, the kit compises the different elements separately and optionally instructions of how to use said ingredients. For example the kit may comprises i) at least one Lamiaceae extract and ii) at least one saponin and optionally instructions of how to use said said ingredients.

In certain embodiments the kit comprises a blend of i) at least one phenolic diterpene (such as carnosic acid and /or carnosol) and/or ii) at least one saponin, and optionally instructions of how to use said ingredients.

In certain embodiments the kit comprises separately i) at least one phenolic diterpene (such as carnosic acid and /or carnosol) and/or ii) at least one saponin, and optionally instructions of how to use said ingredients.

In certain embodiments the kit comprises a blend of at least one saponin (such as a quillaja and/or yucca saponin(s)), a citrus juice (such as lemon juice) and/or hesperidin, and optionally instructions of how to use said ingredients.

In certain embodiments the kit comprises, separately, at least one saponin (such as a quillaja and/or yucca saponin(s)), a citrus juice (such as lemon juice) and/or hesperidin, and optionally instructions of how to use said ingredients.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

When we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of we mean that the extract or composition being described must contain the listed ingredient(s) only. The term “about” as used herein, e.g. when referring to a measurable value (such as an amount or weight of a particular component in the reaction mixture), refers to variations of ±20%, ±10%, ±5%, ±1 %, ±0.5%, or, particularly, ±0.1% of the specified amount.

### EXAMPLES

### Detailed Procedure for Obtaining Rosemary Extracts

The detailed procedure to prepare the composition of Rosemary extract was described in the US Pat. No. 5,859,293 (PCT W096/34534), which is incorporated herein by reference in its entirely. The procedure is briefly summarized as follows: The Rosemary leaves were extracted with acetone at room temperature. After the extraction was completed, the acetone extract was filtered to separate the solution from Rosemary leaves and concentrated under reduced pressure to make concentrated native extract. At this time, the concentrated extract can be dried directly in a vacuum oven under mild heat to make a powdered extract, which is a composition comprising about 15%-30% carnosic acid and 1%-3% carnosol. Or to the concentrated native extract, aqueous sodium carbonate (NaHCCh) was added to dissolve carnosic acid and other organic acids, while base insoluble substances were precipitated out. The solution was filtered to separate from solid, and the filtrate was further concentrated under reduced pressure. Once finishing concentration is achieved, phosphoric acid (H3PO4) was added and the acid insoluble substances (including carnosic acid, carnosol, and carnosic derivatives) were precipitated from the concentrated solution. Through filtering, the precipitated solid was subsequently separated from liquid and rinsed with water to remove impurities. Last, the solid was dried in a vacuum oven and then milled into powder which is a composition containing about 40-85% carnosic acid, 2- 10% carnosol, and 2- 10% 12-O-methylcarnosic acid.

### Salvia Extracts

Salvia officinalis or Salvia apiana leaves were extracted with acetone at reflux (56,4° C.) for 2 hours under mechanical stirring. After the extraction was completed, the acetone extract was filtered to remove the plant residues. The resulting acetonic extract was concentrated under reduced pressure to dryness. The dry extract contains about 10-20% carnosic acid and 1 %-2% carnosol.

### Saponin Extraction From Quillaja Bark or Tree

### Extraction Using Methanol/water

In the case of bark, saponins can be extracted from between 1.2% and 2.2% of dry weight, with an extraction done using 2 g of dried bark and 5017 mL of MeOH: H2O (7/3, v/v) at room temperature for 24 hours.

### Extraction Using Water

Using the common water extraction method, at 50 -60° C. for 3 hours quillaja saponins are extracted at 8.6 to 15.8% when the extraction is done with water in a soxhlet apparatus for 10 hours at a temperature of 100° C. and 6.5 to 8.5% when extracting with water by maceration at room temperature for 24 hours. All saponin contents mentioned above were determined using comparable HPLC methods.

### Saponin Extraction From Quillaja Branches

Saponins may be extracted from quillaja branches using the above methods and any other method from the art.

### Yucca Saponin Extracts

A concentrated 38°Brix Yucca juice concentrate was dried in acidifying conditions, here, in presence of phosphoric acid, and with silica and maltodextrin, as carrier. Any other drying agent or carrier known in the art may be used.

Here used Yucca saponin rich product comprised 9% saponins.

### Hesperidin

Dried immature fruits (Citrus aurantium L.) are exposed to a vapor in order to remove pectins prior to the extraction with water. Subsequently, sodium hydroxide and calcium hydroxide are added in the solution to stabilize the pH value. Filtration steps are used. Following the filtration step, an acidification of the filtrate using HCl is performed. Upon this step the hesperidin precipitates, the liquid solution is removed and the precipitate is dried. The final product (pure hesperidin) contains 90% to 99% hesperidin, preferably more than 95% of hesperidin as measured by HPLC.

### Lemon Juice.

Lemon (Citrus limon L.) juice powder was produced by vacuum drying. One kg of the product is made from around 12.4 kg single strength juice at 8°Brix, obtained from around 21.5 kg of fresh fruit. Lemon juice powder contains fruit solids and silicon dioxide as an anticaking agent.

### Microorganisms

A number of spoilage microorganisms were selected to conduct an in vitro screening (Table 1). During the experiments, the microorganisms were cultured in the presence of different concentrations of the ingredients and growth inhibition was measured. The microorganisms were obtained from TNO’s database or they were ordered. Each strain was cultured in specific conditions of medium and temperature prior to the screening.

TABLE 1

Details on the spoilage microorganisms and on their growing conditions. These were optimized during the preparation phase and applied during the screening phase

| Microorganisms | | | | Incubation | | | |
|---|---|---|---|---|---|---|---|
| Species | Type | Strain | Source | Medium | Temperature | Shaking (rpm) | Duration |
| Lactobacillus plantarum | Bacteria | ATCC 14917 | pickled cabbage | MRSA | 37° C. | 200 | 24h |
| Alicyclobacillus acidoterrestris | Bacteria | DSM 2498 | juice | BAT | 44° C. | 200 | 72h |
| Zygosaccharomyces bailii | Yeast | PYCC 4806 | fermenting rosé wine must | YPD | 30° C. | 200 | 48h |
| Saccharomyces cerevisiae | Yeast | CBS 381 | spoiled beer | YPD | 30° C. | 200 | 24h |
| Schizosaccharomyces pombe | Yeast | DSM 70577 | wine must | MEA | 30° C. | 200 | 48h |
| Dekkera anomala | Yeast | CBS 4210 | cider | MEA | 30° C. | 200 | 48h |
| Aspergillus niger | Fungi | ATCC 16404 | blueberry | MEA | 30° C. | 850 | 6 days |
| Byssochlamys nivea | Fungi | CBS 113246 | pasteurized apple sauce | YPD | 30° C. | 850 | 6 days |

### Incubations

During the experiment, the microorganisms were cultured in the presence of different concentrations of the test ingredients and their growth inhibition was measured by qPCR.

The screening was performed in 96-deep well plates. Each ingredient was tested at 6 concentrations: 1%, 0.5%, 0.25%, 0.125%, 0.061% and 0.031% or lower concentrations. Some ingredients were not easily dissolved despite the use of DMSO. For these ingredients, the concentration range was slightly adjusted to ensure dissolution of the ingredients even at the highest concentration tested.

The following tables describe the different examples and contain information on the exact concentration and the % and range tested for each ingredient. For example on table 3, “extract concentration (%)” refers to the % in the final mixture of quillaja extract or the Rosemary extract. “Carnosic acid (%)” refers to % in the final mixture of carnosic acid (the carnosic acid source is the rosemary extract), “Quillaja saponins (%)” refers to the % in the final mixture of quillaja saponins (being the source of the quillaja saponins the quillaja extract).

TABLE 2

Composition of extracts

| Extract | Carnosic acid (%) | Quillaja saponins (%) |
|---|---|---|
| Rosemary | 41.1 | |
| Quillaja | | 28.59 |
| Quillaja + Rosemary | 20.05 | 14.3 |

Salvia officinalis or Salvia apiana extracts comprise phenolic diterpenes, notably carnosic acid from 5 to 45%.

### Example 1. Quillaja, Rosemary, and Quillaja and Rosemary Antimicrobial Effect on Bacteria

TABLE 3

Quillaja, Rosemary, and Quillaja and Rosemary antimicrobial effect on Lactobacillus plantarum

| Extract | Extract Concentration (%) | Carnosic acid (%) | Quillaja saponins (%) | Expected Lactobacillus plantarum inhibition (%) | Measured Lactobacillus plantarum inhibition (%) |
|---|---|---|---|---|---|
| Quillaja | 1 | | 0.28 | | 1 |
| | 0.5 | | 0.14 | | 0 |
| | 0.25 | | 0.07 | | 2 |
| | 0.125 | | 0.035 | | 1 |
| | 0.061 | | 0.017 | | 1 |
| | 0.031 | | 0.0085 | | -1 |
| Rosemary | 0.55 | 0.225 | | | 83 |
| | 0.275 | 0.113 | | | 6 |
| | 0.138 | 0.056 | | | 2 |
| | 0.069 | 0.028 | | | 4 |
| | 0.034 | 0.014 | | | 5 |
| | 0.017 | 0.007 | | | 3 |
| Quillaja + Rosemary | 0.55 | 0.113 | 0.07 | 6 | 105^{∗} |
| | 0.275 | 0.056 | 0.035 | 2 | 102^{∗} |
| | 0.138 | 0.028 | 0.017 | 4 | 77^{∗} |
| | 0.069 | 0.014 | 0.0085 | 5 | 29^{∗} |
| | 0.034 | 0.007 | 0.004 | 5 | 14^{∗} |
| | 0.017 | 0.003 | 0.002 | NA | 2 |

^{∗}: unexpected effect

When applied alone, rosemary or quillaja extracts could not inhibit Lactobacillus plantarum growth. Only very high concentration in rosemary extract, that could provide 0.22% carnosic acid could inhibit the bacterial growth at 83%. Quillaja extract, even when used at high concentrations failed in inhibiting bacterial growth. Surprisingly when used together, in combination, rosemary and quillaja extract inhibited Lactobacillus planatrum growth partially (from 14%) to fully (105% growth inhibition) when applied at relatively low concentrations: from 0.034% extract concentration, which corresponds to 0.007% carnosic acid and 0.004% quillaja saponins, to 0.275% extract concentration, which corresponds to 0.056% carnosic acid and 0.035% quillaja saponins.

TABLE 4

Salvia antimicrobial effect on Lactobacillus plantarum

| Extract | Extract Concentration (%) | Measured Lactobacillus plantarum inhibition (%) |
|---|---|---|
| Salvia apiana | 0.55 | 100 |
| | 0.275 | 80 |
| | 0.138 | 62 |
| | 0.069 | 43 |
| | 0.034 | 2 |
| | 0.017 | 0 |
| Salvia officinalis | 0.55 | 63 |
| | 0.275 | 6 |
| | 0.138 | 1 |
| | 0.069 | -1 |
| | 0.034 | -1 |
| | 0.017 | 1 |

TABLE 5

Quillaja, Rosemary, and Quillaja and Rosemary antimicrobial effect on Alicyclobacillus acidoterrestris

| Extract | Extract Concentration (%) | Carnosic acid (%) | Quillaja saponins (%) | Expected Alicyclobacillus acidoterrestris inhibition (%) | Measured inhibition Alicyclobacillus acidoterrestris (%) |
|---|---|---|---|---|---|
| Quillaja | 1 | | 0.28 | | 81 |
| | 0.5 | | 0.14 | | -11 |
| | 0.25 | | 0.07 | | -12 |
| | 0.125 | | 0.035 | | -14 |
| | 0.061 | | 0.017 | | -8 |
| | 0.031 | | 0.0085 | | -6 |
| Rosemary | 0.55 | 0.225 | | | 111 |
| | 0.275 | 0.113 | | | 117 |
| | 0.138 | 0.056 | | | 118 |
| | 0.069 | 0.028 | | | 118 |
| | 0.034 | 0.014 | | | 117 |
| | 0.017 | 0.007 | | | 114 |
| | 0.009 | 0.004 | | | 3 |
| | 0.004 | 0.002 | | | -4 |
| | 0.002 | 0.001 | | | -8 |
| | 0.275 | 0.056 | 0.035 | 118 | 116 |
| | 0.138 | 0.028 | 0.017 | 118 | 117 |
| | 0.069 | 0.014 | 0.0085 | 117 | 123^{∗} |
| | 0.034 | 0.007 | 0.004 | 114 | 124^{∗} |
| | 0.017 | 0.004 | 0.002 | 3.44 | 122^{∗} |
| | 0.009 | 0.002 | 0.001 | -4 | 137^{∗} |
| | 0.004 | 0.001 | 0.0005 | -8 | 135^{∗} |
| | 0.002 | 0.0005 | 0.00025 | 0 | 137^{∗} |
| | 0.001 | 0.00025 | 0.000125 | 0 | 137^{∗} |

^{∗}: unexpected effect

When applied alone quillaja extracts even when used at high concentrations failed in inhibiting could not inhibit Alicyclobacillus acidoterrestris growth. Only high concentration in rosemary extract that could provide between 0.007 and 0.22% of carnosic acid could inhibit the bacterial growth.

Surprisingly when used together, in combination, rosemary and quillaja extract inhibited Alicyclobacillus acidoterrestris fully, even a very low concentrations of carnosic acid (0.00025%) and quillaja (0.000125%).

TABLE 6

Salvia antimicrobial effect on Alicyclobacillus acidoterrestris

| Extract | Extract Concentration (%) | Measured Alicyclobacillus acidoterrestris inhibition (%) |
|---|---|---|
| Salvia apiana | 0.55 | 134 |
| | 0.275 | 130 |
| | 0.138 | 125 |
| | 0.069 | 124 |
| | 0.034 | 124 |
| | 0.017 | 111 |
| Salvia officinalis | 0.55 | 123 |
| | 0.275 | 122 |
| | 0.138 | 120 |
| | 0.069 | 111 |
| | 0.034 | 105 |
| | 0.017 | 17 |

### Example 2. Quillaja, Rosemary, and Quillaja and Rosemary Antimicrobial Effect on Yeast

TABLE 7

Quillaja antimicrobial effect on Saccharomyces cerevisiae

| Extract | Extract Concentration (%) | Quillaja saponins (%) | Measured inhibition Saccharomyces cerevisiae (%) |
|---|---|---|---|
| Quillaja | 1 | 0.28 | 101 |
| | 0.5 | 0.14 | 104 |
| | 0.25 | 0.07 | 112 |
| | 0.125 | 0.035 | 109 |
| | 0.061 | 0.017 | 103 |
| | 0.031 | 0.0085 | 111 |

All concentrations of quillaja extract here tested, fully, for at least 100%, inhibited the growth of Saccharomyces cerevisiae.

TABLE 8

Salvia antimicrobial effect on Saccharomyces cerevisiae

| Extract | Extract Concentration (%) | Measured Saccharomyces cerevisiae inhibition (%) |
|---|---|---|
| Salvia apiana | 0.55 | 99 |
| | 0.275 | 7 |
| | 0.138 | 0 |
| | 0.069 | -7 |
| | 0.034 | 4 |
| | 0.017 | 1 |

TABLE 9

Quillaja, Rosemary, and Quillaja and Rosemary antimicrobial effect on Saccharomyces pombe

| Extract | Extract Concentration (%) | Carnosic acid (%) | Quillaja saponins (%) | Expected Saccharomyces pombe inhibition (%) | Measured Saccharomyces pombe inhibition (%) |
|---|---|---|---|---|---|
| Quillaja | 1 | | 0.28 | | 105 |
| | 0.5 | | 0.14 | | 66 |
| | 0.25 | | 0.07 | | 57 |
| | 0.125 | | 0.035 | | 49 |
| | 0.061 | | 0.017 | | 25 |
| | 0.031 | | 0.0085 | | 2 |
| Rosemary | 0.55 | 0.225 | | | 66 |
| | 0.275 | 0.113 | | | -8 |
| | 0.138 | 0.056 | | | -13 |
| | 0.069 | 0.028 | | | -6 |
| | 0.034 | 0.014 | | | -5 |
| | 0.017 | 0.007 | | | -3 |
| Quillaja + Rosemary | 0.55 | 0.113 | 0.07 | 49 | 84^{∗} |
| | 0.275 | 0.056 | 0.035 | 36 | 106^{∗} |
| | 0.138 | 0.028 | 0.017 | 19 | 88^{∗} |
| | 0.069 | 0.014 | 0.0085 | -3 | 87^{∗} |
| | 0.034 | 0.007 | 0.004 | <-1 | 17^{∗} |

^{∗}: unexpected effect.

When applied alone quillaja extracts only used at high concentrations had an effect inhibiting Measured Saccharomyces pombe growth. Only very high concentration in rosemary extract that could provide about 0.28% of carnosic acid could inhibit the yeast growth.

Surprisingly when used together, in combination, rosemary and quillaja extract inhibited Saccharomyces pombe fully, even a very low concentrations of carnosic acid (0.014 %) and quillaja (0.0085%). This is synergy.

TABLE 10

Salvia antimicrobial effect on Saccharomyces pombe.

| Extract | Extract Concentration (%) | Measured Saccharomyces pombe inhibition (%) |
|---|---|---|
| Salvia apiana | 0.55 | 94 |
| | 0.275 | 85 |
| | 0.138 | -1 |
| | 0.069 | -12 |
| | 0.034 | -8 |
| | 0.017 | -6 |

TABLE 11

Quillaja, Rosemary, and Quillaja and Rosemary antimicrobial effect on Dekkera anomala

| Extract | Extract Concentration (%) | Carnosic acid (%) | Quillaja saponins (%) | Expected Dekkera anomala inhibition (%) | Measured Dekkera anomala inhibition (%) |
|---|---|---|---|---|---|
| Quillaja | 1 | | 0.28 | | -13 |
| | 0.5 | | 0.14 | | -2 |
| | 0.25 | | 0.07 | | -8 |
| | 0.125 | | 0.035 | | -7 |
| | 0.061 | | 0.017 | | 9 |
| | 0.031 | | 0.0085 | | 3 |
| Rosemary | 0.55 | 0.225 | | | 98 |
| | 0.275 | 0.113 | | | 1 |
| | 0.138 | 0.056 | | | 13 |
| | 0.069 | 0.028 | | | 6 |
| | 0.034 | 0.014 | | | 15 |
| | 0.017 | 0.007 | | | 3 |
| Quillaja + Rosemary | 0.55 | 0.113 | 0.07 | -7 | 90^{∗} |
| | 0.275 | 0.056 | 0.035 | 6 | 82^{∗} |
| | 0.138 | 0.028 | 0.017 | 7.5 | 88^{∗} |
| | 0.069 | 0.014 | 0.0085 | 12 | 85^{∗} |
| | 0.034 | 0.007 | 0.004 | 3 | 89^{∗} |
| | 0.017 | 0.003 | 0.002 | <3 | 93^{∗} |

^{∗}: unexpected effect

When applied alone quillaja extracts or rosemary extract failed to inhibit the yeast Dekkera anomala growth.

Surprisingly when used together, in combination, rosemary and quillaja extract inhibited Dekkera anomala fully, even a very low concentrations of carnosic acid (0.003%) and quillaja (0.002%). This is synergy.

TABLE 12

Salvia antimicrobial effect on Dekkera anomala

| Extract | Extract Concentration (%) | Measured Dekkera anomala inhibition (%) |
|---|---|---|
| Salvia apiana | 0.55 | 111 |
| | 0.275 | 112 |
| | 0.138 | 81 |
| | 0.069 | 15 |
| | 0.034 | 14 |
| | 0.017 | 16 |
| Salvia officinalis | 0.55 | 97 |
| | 0.275 | 85 |
| | 0.138 | 18 |
| | 0.069 | 19 |
| | 0.034 | 13 |
| | 0.017 | 13 |

Salvia officinalis or Salvia apiana extracts comprise phenolic diterpenes, notably carnosic acid from 5 to 45%.

### Example 3. Yucca, Rosemary, and Yucca and Rosemary Antimicrobial Effect on bacteria Incubations

During the experiment, the microorganisms were cultured in the presence of different concentrations of the test ingredients and their growth inhibition was measured by qPCR.

The screening was performed in 96-deep well plates. Each ingredient was tested at 6 concentrations: (such as 1%, 0.5%, 0.25%, 0.125%, 0.061% and 0.031% or lower concentrations). Some ingredients were not easily dissolved despite the use of DMSO. For these ingredients, the concentration range was slightly adjusted to ensure dissolution of the ingredients even at the highest concentration tested.

TABLE 13

Composition of extracts

| Extract | Carnosic acid (%) | Yucca saponins (%) |
|---|---|---|
| Rosemary | 41.1 | |
| Yucca | | 9 |
| Yucca + Rosemary | 20.05 | 4.5 |

TABLE 14

Yucca, Rosemary and Yucca and Rosemary antimicrobial effect on Lactobacillus plantarum

| Extract | Extract Concentration (%) | Carnosic acid (%) | Yucca saponins (%) | Expected Lactobacillus plantarum inhibition (%) | Measured Lactobacillus plantarum inhibition (%) |
|---|---|---|---|---|---|
| Yucca | 1 | | 0.09 | | 1 |
| | 0.5 | | 0.045 | | 0 |
| | 0.25 | | 0.023 | | 1 |
| | 0.125 | | 0.017 | | 3 |
| | 0.061 | | 0.008 | | 0 |
| | 0.031 | | 0.004 | | 2 |
| Rosemary | 0.55 | 0.225 | | | 83 |
| | 0.275 | 0.113 | | | 6 |
| | 0.138 | 0.056 | | | 2 |
| | 0.069 | 0.028 | | | 4 |
| | 0.034 | 0.014 | | | 5 |
| | 0.017 | 0.007 | | | 3 |
| Yucca + Rosemary | 0.55 | 0.113 | 0.045 | 6 | 81^{∗} |
| | 0.275 | 0.056 | 0.023 | 3 | 25^{∗} |
| | 0.138 | 0.028 | 0.017 | 7 | 1 |
| | 0.069 | 0.014 | 0.008 | 5 | 3 |
| | 0.034 | 0.007 | 0.004 | 5 | -4 |
| | 0.017 | 0.003 | 0.002 | <5 | -2 |

^{∗}: unexpected effect

When applied alone, rosemary or Yucca extracts could not inhibit Lactobacillus plantarum growth. Only very high concentration in rosemary extract, that could provide 0.22% carnosic acid, could inhibit the bacterial growth at 83%. Yucca extract, even when used at high concentrations failed in inhibiting bacterial growth. Surprisingly when used together, in combination, rosemary and yucca extract inhibited Lactobacillus planatrum growth partially (from 25%) to strongly (81% growth inhibition) when applied at relatively low concentrations: from 0.275% extract concentration, which corresponds to 0.056% carnosic acid and 0.023% yucca saponins, to 0.55% extract concentration, which corresponds to 0.113% carnosic acid and 0.045% yucca saponins. This is synergy.

TABLE 15

Salvia antimicrobial effect on Lactobacillus plantarum

| Extract | Extract Concentration (%) | Measured Lactobacillus plantarum inhibition (%) |
|---|---|---|
| Salvia apiana | 0.55 | 100 |
| | 0.275 | 80 |
| | 0.138 | 62 |
| | 0.069 | 43 |
| | 0.034 | 2 |
| | 0.017 | 0 |
| Salvia officinalis | 0.55 | 63 |
| | 0.275 | 6 |
| | 0.138 | 1 |
| | 0.069 | -1 |
| | 0.034 | -1 |
| | 0.017 | 1 |

TABLE 16

Yucca, Rosemary and Yucca and Rosemary antimicrobial effect on Alicyclobacillus acidoterrestris

| Extract | Extract Concentration (%) | Carnosic acid (%) | Yucca saponins (%) | Expected Alicyclobacillus acidoterrestris inhibition (%) | Measured inhibition Alicyclobacillus acidoterrestris (%) |
|---|---|---|---|---|---|
| Yucca | 1 | | 0.09 | | 120 |
| | 0.5 | | 0.045 | | 125 |
| | 0.25 | | 0.023 | | 119 |
| | 0.125 | | 0.017 | | 34 |
| | 0.061 | | 0.008 | | 12 |
| | 0.031 | | 0.004 | | 0 |
| Rosemary | 0.55 | 0.225 | | | 111 |
| | 0.275 | 0.113 | | | 117 |
| | 0.138 | 0.056 | | | 118 |
| | 0.069 | 0.028 | | | 118 |
| | 0.034 | 0.014 | | | 117 |
| | 0.017 | 0.007 | | | 114 |
| | 0.009 | 0.004 | | | 3 |
| Rosemary + Yucca | 0.55 | 0.113 | 0.045 | 121 | 116 |
| | 0.275 | 0.056 | 0.023 | 119 | 128^{∗} |
| | 0.138 | 0.028 | 0.017 | 118 | 130^{∗} |
| | 0.069 | 0.014 | 0.008 | 117 | 125^{∗} |
| | 0.034 | 0.007 | 0.004 | 114 | 118^{∗} |
| | 0.017 | 0.004 | 0.002 | 0 | 114^{∗} |
| | 0.009 | 0.002 | 0.001 | 0 | 139^{∗} |
| | 0.004 | 0.001 | 0.0005 | 0 | 135^{∗} |
| | 0.002 | 0.0005 | 0.00025 | 0 | 5^{∗} |

^{∗}: unexpected effect

When applied alone, rosemary or Yucca extracts could inhibit Alicyclobacillus acidoterrestris growth only at high concentration. Surprisingly when used together, in combination, rosemary and yucca extract inhibited Alicyclobacillus acidoterrestris growth when applied at relatively low concentrations (for example 0.0005% carnosic acid and 0.00025% yucca saponins). This is synergy.

TABLE 16

Salvia antimicrobial effect on Alicyclobacillus acidoterrestris

| Extract | Extract Concentration (%) | Measured Alicyclobacillus acidoterrestris inhibition (%) |
|---|---|---|
| Salvia apiana | 0.55 | 134 |
| | 0.275 | 130 |
| | 0.138 | 125 |
| | 0.069 | 124 |
| | 0.034 | 124 |
| | 0.017 | 111 |
| Salvia officinalis | 0.55 | 123 |
| | 0.275 | 122 |
| | 0.138 | 120 |
| | 0.069 | 111 |
| | 0.034 | 105 |
| | 0.017 | 17 |

### Example 4: Yucca, Rosemary, Salvia and Yucca and Rosemary Antimicrobial Effect on Yeast

TABLE 17

Yucca antimicrobial effect on Zygosaccharomyces baillii

| Extract | Extract Concentration (%) | Yucca saponins (%) | Measured inhibition Zygosaccha-romyces baillii (%) |
|---|---|---|---|
| Yucca | 1 | 0.09 | 97 |
| | 0.5 | 0.045 | 93 |
| | 0.25 | 0.023 | 106 |
| | 0.125 | 0.017 | 93 |
| | 0.061 | 0.008 | 92 |
| | 0.031 | 0.004 | 111 |

All concentrations of yucca extract here tested, fully, for at least 92%, inhibited the growth of Zygosaccharomyces baillii.

TABLE 18

Yucca antimicrobial effect on Saccharomyces cerevisiae:

| Extract | Extract Concentration (%) | Yucca saponins (%) | Measured inhibition Saccharomyces cerevisiae (%) |
|---|---|---|---|
| Yucca | 1 | 0.09 | 106 |
| | 0.5 | 0.045 | 122 |
| | 0.25 | 0.023 | 103 |
| | 0.125 | 0.017 | 112 |
| | 0.061 | 0.008 | 113 |
| | 0.031 | 0.004 | 99 |

All concentrations of yucca extract here tested, fully, for at least 99%, inhibited the growth of Saccharomyces cerevisiae.

TABLE 19

Salvia antimicrobial effect on Saccharomyces cerevisiae

| Extract | Extract Concentration (%) | Measured Saccharomyces cerevisiae inhibition (%) |
|---|---|---|
| Salvia apiana | 0.55 | 99 |
| Salvia officinalis | 0.55 | 20 |

TABLE 20

Yucca and Rosemary antimicrobial effect on Saccharomyces pombe

| Extract | Extract Concentration (%) | Carnosic acid (%) | Yucca saponins (%) | Measured Saccharomyces pombe inhibition (%) |
|---|---|---|---|---|
| Yucca | 1 | | 0.09 | 121 |
| | 0.5 | | 0.045 | 114 |
| | 0.25 | | 0.023 | 109 |
| | 0.125 | | 0.017 | 110 |
| | 0.061 | | 0.008 | 115 |
| | 0.031 | | 0.004 | 113 |
| Rosemary | 0.55 | 0.225 | | 66 |
| | 0.275 | 0.113 | | -8 |
| | 0.138 | 0.056 | | -13 |
| | 0.069 | 0.028 | | -6 |
| | 0.034 | 0.014 | | -5 |
| | 0.017 | 0.007 | | -3 |

All concentrations of yucca extract here tested inhibited the growth of Saccharomyces pombe.

TABLE 21

Salvia antimicrobial effect on Saccharomyces pombe

| Extract | Extract Concentration (%) | Measured Saccharomyces pombe inhibition (%) |
|---|---|---|
| Salvia apiana | 0.55 | 94 |
| | 0.275 | 85 |
| | 0.138 | -1 |
| | 0.069 | -12 |

### Example 5. Yucca, Rosemary, Salvia and Yucca and Rosemary Antimicrobial Effect on Dekkera Anomala

TABLE 22

Yucca, Rosemary and Yucca and Rosemary antimicrobial effect on Dekkera anomala

| Extract | Extract Concentration (%) | Carnosic acid (%) | Yucca saponins (%) | Expected Dekkera anomala inhibition (%) | Measured Dekkera anomala inhibition (%) |
|---|---|---|---|---|---|
| Yucca | 1 | | 0.09 | | -9 |
| | 0.5 | | 0.045 | | -2 |
| | 0.25 | | 0.023 | | -7 |
| | 0.125 | | 0.017 | | -1 |
| Rosemary | 0.55 | 0.225 | | | 98 |
| | 0.275 | 0.113 | | | 1 |
| | 0.138 | 0.056 | | | 13 |
| | 0.069 | 0.028 | | | 6 |
| Yucca + Rosemary | 0.55 | 0.113 | 0.045 | 1 | 91^{∗} |
| | 0.275 | 0.056 | 0.023 | 6 | 93^{∗} |
| | 0.138 | 0.028 | 0.017 | 5 | 61^{∗} |
| | 0.069 | 0.014 | 0.008 | 8 | 6 |

^{∗}: unexpected effect

When applied alone Yucca extracts could not inhibit Dekkera anomala growth. Rosemary extract only shown an inhibition at very high concentration. Surprisingly when used together, in combination, rosemary and yucca extract inhibited Dekkera anomala growth.

TABLE 23

Salvia antimicrobial effect on Dekkera anomala

| Extract | Extract Concentration (%) | Measured Dekkera anomala inhibition (%) |
|---|---|---|
| Salvia apiana | 0.55 | 111 |
| | 0.275 | 112 |
| | 0.138 | 81 |
| | 0.069 | 15 |
| | 0.034 | 14 |
| | 0.017 | 16 |
| Salvia officinalis | 0.55 | 97 |
| | 0.275 | 85 |
| | 0.138 | 18 |
| | 0.069 | 19 |
| | 0.034 | 13 |
| | 0.017 | 13 |

### Example 6. Quillaja, Rosemary,Yucca, Quillaja and Rosemary, Yucca and Rosemary, and Yucca and Rosemary and Quillaja Antimicrobial Effect on Yeasts

TABLE 24

Yucca, Rosemary, Salvia, Quillaja, Yucca and Rosemary, Quillaja and Rosemary, and Yucca and Quillaja and Rosemary antimicrobial effect on Dekkera anomala

| Extract | Extract Concentration (%) | Carnosic acid (%) | Yucca saponins (%) | Quillaja saponins | Expected Dekkera anomala inhibition (%) | Measured Dekkera anomala inhibition (%) |
|---|---|---|---|---|---|---|
| Yucca | 1 | | 0.09 | | | -13 |
| | 0.5 | | 0.045 | | | -2 |
| | 0.25 | | 0.023 | | | -8 |
| | 0.125 | | 0.017 | | | -7 |
| | 0.061 | | 0.008 | | | 9 |
| | 0.031 | | 0.004 | | | -4 |
| Rosemary | 0.55 | 0.225 | | | | 98 |
| | 0.275 | 0.113 | | | | 1 |
| | 0.138 | 0.056 | | | | 13 |
| | 0.069 | 0.028 | | | | 6 |
| | 0.034 | 0.014 | | | | 15 |
| | 0.017 | 0.007 | | | | 3 |
| Yucca + Rosemary | 0.55 | 0.113 | 0.045 | | 1 | 91^{∗} |
| | 0.275 | 0.056 | 0.023 | | 6 | 93^{∗} |
| | 0.138 | 0.028 | 0.017 | | 5 | 61^{∗} |
| | 0.069 | 0.014 | 0.008 | | 8 | 6 |
| | 0.034 | 0.007 | 0.004 | | 0 | -3 |
| | 0.017 | 0.003 | 0.002 | | <0 | 2 |
| Quillaja | 1 | | | 0.28 | | -9 |
| | 0.5 | | | 0.14 | | -2 |
| | 0.25 | | | 0.07 | | -7 |
| | 0.125 | | | 0.035 | | -1 |
| | 0.061 | | | 0.017 | | 0 |
| | 0.031 | | | 0.0085 | | -3 |
| Quillaja + Rosemary | 0.55 | 0.113 | | 0.07 | -7 | 90^{∗} |
| | 0.275 | 0.056 | | 0.035 | 6 | 82^{∗} |
| | 0.138 | 0.028 | | 0.017 | 7.5 | 88^{∗} |
| | 0.069 | 0.014 | | 0.0085 | 12 | 85^{∗} |
| | 0.034 | 0.007 | | 0.004 | 3 | 89^{∗} |
| | 0.017 | 0.003 | | 0.002 | <3 | 93^{∗} |
| Quillaja+ Rosemary+ Yucca | 1 | 0.113 | 0.023 | 0.07 | -4 | 129^{∗} |
| | 0.5 | 0.056 | 0.017 | 0.035 | 1.5 | 126^{∗} |
| | 0.25 | 0.028 | 0.008 | 0.017 | 5 | 133^{∗} |
| | 0.125 | 0.014 | 0.004 | 0.0085 | 2.5 | 112^{∗} |
| | 0.061 | 0.007 | 0.002 | 0.0043 | <-1 | 19^{∗} |
| | 0.031 | 0.003 | 0.001 | 0.0022 | -1 | 7* |

^{∗}: unexpected effect

When applied alone Yucca extracts or quillaja extracts could not inhibit Dekkera anomala growth. Rosemary extract only shown an inhibition at very high concentration. Surprisingly when used together, in combination, rosemary, yucca and quillja extract inhibited Dekkera anomala growth even a very low concentrations.

Unexpected improvement of ternary combination Quillaja/ yucca/rosemary over quilla-ja/rosemary and yucca/ rosemary : full inhibition (>100%) for very low concentrations: from 0.008% quillaja saponins, 0.004% yucca saponins and 0.014% carnosic acid.

### Example 7. Yucca Antimicrobial Effect on Mold

TABLE 25

Yucca effect on Byssochlamys nivea

| Extract | Extract Concentration (%) | Yucca saponins (%) | Measured Byssochlamys nivea inhibition (%) |
|---|---|---|---|
| Yucca | 1 | 0.09 | 88 |
| | 0.5 | 0.045 | 98 |
| | 0.25 | 0.023 | 97 |
| | 0.125 | 0.017 | 98 |
| | 0.061 | 0.008 | 107 |
| | 0.031 | 0.004 | 110 |

### Example 8. Antimicrobial Effect in Food Matrices of the Compositions of the Invention,

Fish matrix was made of fish and water (50/50), without preservatives. The matrix was homogenized in a blade mixer grinder and then sterilized at 121° C. for 20 minutes, prior the tests.

### Methods

Blends were added at 0.5 % into the matrix. Matrices without preservatives (0.5 % of NaCl 0.9%) and matrices with conventional chemical preservatives (0.3% of sodium lactate or potassium nitrite, separately) were used as negative and positive control, respectively.

Each matrix type was portioned into sterile flasks and inoculated with 3 Log CFU/g of different microorganisms, separately. Each flask corresponds to one sample at a given date of analysis.

The inoculated matrices were incubated at 25° C.

The counting of the microorganisms was determined in specific agar medium.

TABLE 26

Results of microorganisms counting in fish model, expressed in Log CFU/g as average of two determinations.

| | | Bacillus subtillis totaux (vegetative cells + spores) | Bacillus subtillis spores (Bacillus subtillis totaux - Bacillus subtillis vegetative cells) |
|---|---|---|---|
| | D0 | D14 | D14 |
| Negative control, without preservative | 3.89 | 3.04 | 3.51 |
| Positive control, Sodium lactate at 0.3% | | 2.90 | 2.35 |
| Positive control, Potassium nitrite at 0.3% | | 2.60 | 2.39 |
| 0.25% of Rosemary/ 0.25% of Quil-laia | | 2.65 | 2.69 |
| 0.15 % of Yucca/ 0.2% of citrus juice / 0.15% of citrus hesperidin | | 2.39 | 2.48 |

1. C] A composition comprising at least one of:
a) at least one Lamiaceae extract; or
b) at least one saponin.

2. C] The composition according to claim 1, wherein the Lamiaceae extract is selected from rosemary extract, thyme extract, mint extract, salvia extract, Lepechinia extract, Oreganum extract, [, ]Hyssopus extract, and mixtures thereof.

3. C] The composition according to claim 1, wherein the Lamiaceae extract comprises phenolic diterpenes, or is enriched in phenolic diterpenes, or comprises phenolic diterpenes and is enriched with phenolic diterpenes.

4. The composition according to claim 3, wherein the Lamiaceae extractcomprises at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90%, at least about 95%, or at least about 99 wt % of one or more phenolic diterpenes.

5. C] The composition according to claim 4, wherein the ratio[n] between said phenolic diterpen(s) and the at least one saponin is from about 30:1 to 1:30.

6. C] The composition according to claim 4, comprising at least one of:
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition (w/w) of carnosic acid;
b) from about 1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition (w/w).

7. C] A composition comprising at least one of:
a) at least one saponin; or
b) at least one phenolic diterpene.

8. C] The composition according to claim 7, wherein the ratio[n] between said phenolic diterpen(s) and the at least one saponin is from about 30:1 to 1:30.

9. C] The composition according to claim 8, wherein the phenolic diterpene is selected from carnosic acid, carnosol, methylcarnosate, rosmanol, isorosmanol, 1 1, 12 -di-O-methylisorosmanol, 12-O-methylcarnosic acid, rosmanol-9-ethyl ether, circimaritin, Methylated monooxidized product of carnosic acid, genkwanin, epirosmanol, epiisorosmanol, carnosic acid derivative, epirosmanol ethyl ether, cryptotanshinone and mixtures thereof.

10. C] The composition according to claim 9, wherein the phenolic diterpene is at least one of carnosic acid or carnosol.

11. C] The composition according to claim 10, wherein at least one of the carnosic acid or carnosol is derived from Lamiaceae.

12. C] The composition according to claim 11, comprising at least one of:
a) from about 1%, 5%, 10%, 15%, 20%, 25%, 30%, 35%, 40% or 45% to about 95%, 90%, 85%, 80%, 75%, 70%, 65%, 60%, 55% or 50% by weight of the final composition(w/w) of carnosic acid; or
b) from about [ ]1%, 5%, 10%, 15%, 20%, 25%, 30%, 35%, 40% or 45% to about 95%, 90%, 85%, 80%, 75%, 70%, 65%, 60%, 55% or 50% of carnosol by weight of the final composition(w/w.

13. C] The composition according to claim 1, wherein the at least one saponin is obtained or obtainable from soya, beans, peas, oat, Solanum and Allium species, tomato, asparagus, tea, peanut, spinach, sugar beet, yam, blackberry, liquorice root, primula root, senega root, Quillaja, Yucca,Gyposphila, and mixtures thereof.

14. C] The composition according to claim 13, wherein the saponin(s) comprise at least one of quillajaor yucca saponin(s).

15. C] The composition according to claim 1, wherein the at least one saponin is at a concentration of at least about 0.001%, 0.01%, 0.1%, 1%, 2%, 3%, 4%, 5%, 6%, 7%, 8%, 9%, 10%, 15%, 20%, 30%, 40%, 50%, or 60% w/w (by weight of the final composition w/w).

16. A food or beverage product for humans or animals, a nutritional supplement, nutraceutical formulation, a dietary, a sports supplement, a fragrance or flavouring, a pharmaceutical or veterinary formulation, an oenological or cosmetic formulation comprising a composition according to claim 1.

17. The product according to claim 16, wherein the concentration of phenolic diterpene(s)in the final product is from 12 ppm, 15 ppm, 20 ppm, 30 ppm, 40 ppm, 50 ppm, 60 ppm, 70 ppm, 80 ppm, 90 ppm, 100 ppm, to about 40%, 30%, 20%, 10%, 9%, 8%, 7%, 6%, 5%, 4%, 4%, 3%, 2%, 1% w/w (10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm.

18. The product according to claim 16, wherein the concentration of saponin(s) in the final product is from about 12 ppm, 15 ppm, 20 ppm, 30 ppm, 40 ppm, 50 ppm, 60 ppm, 70 ppm, 80 ppm, 90 ppm, 100 ppm, to about 40%, 30%, 20%, 10%, 9%, 8%, 7%, 6%, 5%, 4%, 4%, 3%, 2%, 1% w/w (10000 ppm), 9000 ppm, 8000 ppm, 7000 ppm, 6000 ppm, 5000 ppm, 4000 ppm, 3000 ppm, 2000 ppm, 1000 ppm, 800 ppm, 600 ppm, 500 ppm, 400 ppm, 300 ppm to 200 ppm.

19. C] A composition comprising at least one saponin(s)[,] and at least one of a citrus juice or extract or hesperidin.

20. A food or beverage product for humans or animals, a nutritional supplement, nutraceutical formulation, a dietary, a sports supplement, a fragrance or flavouring, a pharmaceutical or veterinary formulation, an oenological or cosmetic formulation comprising a composition according to claim 19.

21. (canceled)

22. (canceled)

23. (canceled)

24. (canceled)

25. (canceled)

26. A method for processing a food or beverage product for humans or animals, a nutritional supplement, a nutraceutical formulation, a fragrance or flavouring, a pharmaceutical or veterinary formulation, an oenological or cosmetic formulation comprising: applying to or incorporating into a food or beverage product for humans or animals, a nutritional supplement, a nutraceutical formulation, a fragrance or flavouring, a pharmaceutical or veterinary formulation, an oenological or cosmetic formulation, a composition according to claim 1.

27. The method according to claim 26, wherein applying to or incorporating into the product comprises applying the composition in the form of a dry powder or a liquid form to the product.

28. The method according to claim 27, wherein the product is a food selected from meat, fish and poultry, a beverages and emulsified or non-emulsified sauces.

29. The method according to any of claim 28, wherein the method extends the color life, inhibits, decreases or limits at least one of bacterial, yeast or mold growth in the product.

30. The method according to claim 29, wherein the bacterial growth inhibited, decreased limited is selected acid-lactic bacteria; thermo-acidophilic bacteria, Xerophilic bacteria, acidophilic bacteria,and combinations thereof.

31. The method according to claim 29, wherein the yeast growth inhibited, decreased[s] or limited is selected from the genus, Candida, Saccharomyces, DekkeraBrettanomyces, Zygosaccharomyces and combinations thereof.

32. The method according to claim 29, wherein the mold growth inhibited, decreased[s] or limited is selected from selected from Byssochlamys genus, Penicillium genus, Aspergillus genus, and combinations thereof.

33. A food or beverage productobtained according to the method of claim 26.

34. A packaged food or beverage product comprising a product according to claim 16, wherein the food is packaged in an atmosphere comprising 20% or more oxygen or in an atmospheric environment.

35. An] The composition of claim 7, wherein the composition is at least one of an antimicrobial, antifungal, or antimold composition comprising an effective amount of a phenolic diterpene and an effective amount of at least one saponin.

36. The composition according to claim 35 wherein the composition is effective against at least one of the following: (i) yeast selected from the group consisting of genus Candida, Saccharomyces, Dekkera, Brettanomyces, Zygosaccharomyces, (ii) bacteria selected from acid-lactic bacteria, thermo-acidophilic bacteria, Xerophilic bacteria, and acidophilic bacteria; or (iii) molds, and (iv) combinations thereof.

37. (canceled)

38. A kit comprising:
a) at least one phenolic diterpene and at least one saponin formulated together or separately[,]; and at least one of
b) at least one Lamiaceae extract and at least one saponin formulated together or separately, or
c) at least one of at least one saponin, a citrus juice,or hesperidin formulated together or separately; and
optionally instructions of how to use said ingredients.

39. The composition of claim 3, wherein the phenolic diterpenes are selected from carnosic acid, carnosol, methylcarnosate, and mixtures thereof.

40. The composition of claim 39, wherein the Lamiaceae extract comprises other phenolic diterpene derivatives, or is enriched with other phenolic diterpene derivatives, or comprises other phenolic diterpene derivatives and is enriched with other phenolic diterpene derivatives, wherein said other phenolic diterpene derivatives are selected from rosmanol, isorosmanol, 1 1, 12 -di-O-methylisorosmanol, 12-O-methylcarnosic acid, rosmanol-9-ethyl ether, circimaritin, Methylated monooxidized product of carnosic acid, genkwanin, epirosmanol, epiisorosmanol, carnosic acid derivative, epirosmanol ethyl ether, cryptotanshinone. and mixtures thereof.

41. The composition according to claim 7, wherein the at least one saponin is obtained or obtainable from soya, beans, peas, oat, Solanum and Allium species, tomato, asparagus, tea, peanut, spinach, sugar beet, yam, blackberry, liquorice root, primula root, senega root, Quillaja, Yucca, Gyposphila, and mixtures thereof.

42. The composition according to claim 7, wherein the at least one saponin is at a concentration of at least about 0.001%, 0.01%, 0.1%, 1%, 2%, 3%, 4%, 5%, 6%, 7%, 8%, 9%, 10%, 15%, 20%, 30%, 40%, 50%, 60% % w/w (by weight of the final composition w/w).

43. A food or beverage product for humans or animals, a nutritional supplement, nutraceutical formulation, a dietary, a sports supplement, a fragrance or flavouring, a pharmaceutical or veterinary formulation, an oenological or cosmetic formulation comprising a composition according to claim 7.

44. A method for processing a food or beverage product for humans or animals, a nutritional supplement, a nutraceutical formulation, a fragrance or flavouring, a pharmaceutical or veterinary formulation, an oenological or cosmetic formulation comprising: applying to or incorporating into a food or beverage product for humans or animals, a nutritional supplement, a nutraceutical formulation, a fragrance or flavouring, a pharmaceutical or veterinary formulation, an oenological or cosmetic formulation, a composition according to claim 7.

45. A method for processing a food or beverage product for humans or animals, a nutritional supplement, a nutraceutical formulation, a fragrance or flavouring, a pharmaceutical or veterinary formulation, an oenological or cosmetic formulation comprising: applying to or incorporating into a food or beverage product for humans or animals, a nutritional supplement, a nutraceutical formulation, a fragrance or flavouring, a pharmaceutical or veterinary formulation, an oenological or cosmetic formulation, a composition according to claim 19.

46. A packaged food or beverage product comprising a product according to claim 33, wherein the food is packaged in an atmosphere comprising 20% or more oxygen or in an atmospheric environment.
